(19) United States
(12) Patent Application Publication
(10) Pub. No.: US 2022/0287966 A1
(43) Pub. Date: Sep. 15, 2022

(54) Stable Liquid Lipid Nanoparticle Formulations

(71) Applicant: Translate Bio, Inc., Lexington, MA (US)

(72) Inventors: Shrirang Karve, Lexington, MA (US); Ashish Sarode, Lexington, MA (US); Natalia Vargas Montoya, Lexington, MA (US); Priyal Patel, Lexington, MA (US); Frank DeRosa, Lexington, MA (US)

(21) Appl. No.: 17/534,956

(22) Filed: Nov. 24, 2021

Related U.S. Application Data
(60) Provisional application No. 63/118,243, filed on Nov. 25, 2020.

(51) Int. Cl. A61K 9/127; A61K 39/00

(52) U.S. Cl. CPC A61K 9/1271; A61K 39/00; A61K 2039/53

(57) ABSTRACT

The present invention provides, among other things, a liquid lipid nanoparticle (LNP) formulation encapsulating mRNA encoding a peptide or polypeptide, that is resistant to aggregation and to mRNA degradation following multiple rounds of freezing at −20° C. and rethawing.

### CROSS-REFERENCE TO RELATED APPLICATIONS

This application claims priority to and benefit of U.S. Provisional Application No. 63/118,243, filed on Nov. 25, 2020, the content of which is hereby incorporated by reference in its entirety.

### BACKGROUND

Nucleic acid-based technologies are increasingly important for various therapeutic applications including, but not limited to, messenger RNA therapy. Efforts to deliver nucleic acids have included the creation of compositions formulated to protect nucleic acids from degradation when delivered in vivo. One type of delivery vehicle for nucleic acids has been lipid nanoparticles. Important parameters to consider for the successful use of lipid nanoparticles as a delivery vehicle include lipid nanoparticle formation, physical properties of lipid components, nucleic acid encapsulation efficiencies, in vivo nucleic acid release potential, and lipid nanoparticle toxicity.

The creation of stable lipid nanoparticles that are resistant to freeze/thaw cycles remains a challenge in the art.

### SUMMARY OF THE INVENTION

The present invention provides, among other things, a liquid lipid nanoparticle (LNP) formulation encapsulating mRNA encoding a peptide or polypeptide that is resistant to aggregation and/or to mRNA degradation following multiple rounds of freezing at −20° C. and rethawing. The inventors surprisingly discovered that LNP formulations having high ionic strength prevents aggregation and/or mRNA degradation of the LNPs following multiple rounds of freezing and thawing. The inventors surprisingly discovered that high ionic strength LNP formulations, which were stable and resistant to aggregation and/or mRNA degradation, could be achieved by either using a higher buffer strength or high salt concentration in the LNP formulation.

In some aspects, a liquid lipid nanoparticle (LNP) formulation is provided encapsulating mRNA encoding a peptide or polypeptide, that is resistant to aggregation and to mRNA degradation, the LNP formulation comprising: a. one or more LNPs having a lipid component comprising or consisting of a cationic lipid, a non-cationic lipid, a PEG-modified lipid and optionally cholesterol; b. mRNA encapsulated within the one or more lipid nanoparticles and encoding a peptide or polypeptide; c. a sugar or a sugar alcohol; d. an LNP formulation pH of from 6.0 to 8.0; e. a pH buffer that at a minimum buffered ionic strength provides the LNP formulation pH; f. optionally one or more additional agents that provide ionic strength to the LNP formulation; wherein a total concentration of pH buffer from (e.), and optionally one or more additional agents from (f.), provide(s) an ionic strength of the LNP formulation that is at least two times greater than the minimum buffered ionic strength; wherein following three rounds of freezing at −20° C. and rethawing, the LNP formulation exhibits (i) less aggregation, (ii) less degradation of the encapsulated mRNA, or (iii) both (i) and (ii), as compared to an identical LNP formulation that has only the minimum buffered ionic strength in the LNP formulation instead of an ionic strength that is at least two times greater than the minimum buffered ionic strength.

In some embodiments, the LNP formulation comprises one or more cryoprotectants. The cryoprotectants can be penetrating or non-penetrating. For example, in some embodiments, the penetrating cryoprotectants comprises glycerol, ethylene glycol, tri-ethylene glycol, propylene glycol, or tetra-ethylene glycol. Accordingly, in some embodiments, the penetrating cryoprotectants comprises glycerol. In some embodiments, the penetrating cryoprotectant comprises ethylene glycol. In some embodiments, the penetrating cryoprotectant comprises tri-ethylene glycol. In some embodiments, the penetrating cryoprotectant comprises propylene glycol. In some embodiments, the penetrating cryoprotectant comprises tetra-ethylene glycol.

In some embodiments, the non-penetrating cyroproctants are selected from sugars and/or polymers. For example, in some embodiments, the non-penetrating cryoprotectants are selected from one or more of the following sugars: dextrose, sorbitol, trehalose, sucrose, raffinose, dextran, or inulin. Accordingly, in some embodiments, the non-penetrating cryoprotectants comprises dextrose. In some embodiments, the non-penetrating cryoprotectants comprises sorbitol. In some embodiments, the non-penetrating cryoprotectants comprises trehalose. In some embodiments, the non-penetrating cryoprotectants comprises sucrose. In some embodiments, the non-penetrating cryoprotectants comprises raffinose. In some embodiments, the non-penetrating cryoprotectants comprises dextran. In some embodiments, the non-penetrating cryoprotectants comprises inulin.

In some embodiments, the non-penetrating cryoprotectants are selected from one or more of the following polymers: PVP, PVA, Poloxamer, or PEG. Accordingly, in some embodiments, the non-penetrating cryoprotectants are selected from PVP. In some embodiments, the non-penetrating cryoprotectants are selected from Poloxamer. In some embodiments, the non-penetrating cryoprotectants are selected from PEG.

In some embodiments, a method of making a stable liquid solution of mRNA in an LNP is provided. For example, in some embodiments, the mRNA encapsulated in the LNPs is produced by in vitro transcription (IVT). In some embodiments, the mRNA is synthesized using a suitable RNA polymerase, such as SP6 RNA polymerase. Accordingly, in some embodiments, the mRNA is synthesized using SP6 RNA polymerase. The LNPs comprise, for example, a cationic lipid, a non-cationic lipid, a PEG-modified lipid and optionally cholesterol

In some embodiments, the non-cationic lipid is selected from 1,2-Dierucoyl-sn-glycero-3-phosphoethanolamine (DEPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, or 1-stearoyl-2-oleoyl-phosphatidyethanolamine (SOPE).

In some embodiments, the non-cationic lipid is at a molar ratio of greater than 10%. For example, in some embodiments, the non-cationic lipid is at a lipid molar ratio of 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%. In some embodiments, the non-cationic lipid is at a lipid molar ratio of about 15%. In some embodiments, the non-cationic lipid is at a lipid molar ratio of about 20%. In some embodiments, the non-cationic lipid is at a lipid molar ratio of about 25%. In some embodiments, the non-cationic lipid is at a lipid molar ratio of about 30%. In some embodiments, the non-cationic lipid is at a lipid molar ratio of about 35%. In some embodiments, the non-cationic lipid is at a lipid molar ratio of about 40%. In some embodiments, the non-cationic lipid is at a lipid molar ratio of about 45%. In some embodiments, the non-cationic lipid is at a lipid molar ratio of about 50%.

In some embodiments, the non-cationic lipid is dioleoylphosphatidylethanolamine (DOPE).

In some embodiments, the DOPE is at a lipid molar ratio of greater than 10%. For example, in some embodiments, the DOPE is at a lipid molar ratio of 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%. In some embodiments, the DOPE is at a lipid molar ratio of about 15%. In some embodiments, the DOPE is at a lipid molar ratio of about 20%. In some embodiments, the DOPE is at a lipid molar ratio of about 25%. In some embodiments, the DOPE is at a lipid molar ratio of about 30%. In some embodiments, the DOPE is at a lipid molar ratio of about 35%. In some embodiments, the DOPE is at a lipid molar ratio of about 40%. In some embodiments, the DOPE is at a lipid molar ratio of about 45%. In some embodiments, the DOPE is at a lipid molar ratio of about 50%. In some embodiments, the DOPE is at a lipid molar ratio of between about 10% and 30%.

In some embodiments, the cationic lipid is a lipidoid. In some embodiments, the lipidoid is at a molar ratio of about, for example, 40%-60%. In some embodiments, the lipidoid is at a molar ratio of about 50%-60%. In some embodiments, the lipidoid is at a molar ratio of about 40%. In some embodiments, the lipidoid is at a molar ratio of about 50%. In some embodiments, the lipidoid is at a molar ratio of about 60%.

In some embodiments, the mRNA encodes a protein deficient in a subject. For example, in some embodiments, the protein deficient in a subject is CFTR.

In some embodiments, the mRNA encodes a vaccine antigen. For example, in some embodiments, the vaccine antigen is a SARS-CoV-2 antigen.

In some embodiments, the sugar is a disaccharide. In some embodiments, the disaccharide is trehalose.

In some embodiments, the sugar or sugar alcohol is selected from the group consisting of dextrose, sorbitol, trehalose, sucrose, raffinose, dextran, and inulin. Accordingly, in some embodiments, the sugar or sugar alcohol is dextrose. In some embodiments, the sugar or sugar alcohol is sorbitol. In some embodiments, the sugar or sugar alcohol is trehalose. In some embodiments, the sugar or sugar alcohol is sucrose. In some embodiments, the sugar or sugar alcohol is raffinose. In some embodiments, the sugar or sugar alcohol is dextran. In some embodiments, the sugar or sugar alcohol is inulin.

In some embodiments, the trehalose is at a concentration of between about 1%-20%. In some embodiments, the trehalose is at a concentration of between about 2.5%-3.0%. In some embodiments, the trehalose is at a concentration of between about 5.0%-15%. In some embodiments, the trehalose is at a concentration of between about 10%-20%.

In some embodiments, the pH is between about 6.0 and about 8.0. For example, in some embodiments, the pH is between about 6.0-7.0, 6.5-7.5 or 7.0-8.0. Accordingly, in some embodiments, the pH is between about 6.0-7.0. In some embodiments, the pH is between about 6.5-7.5. In some embodiments, the pH is between about 7.0-8.0. In some embodiments, the pH is about 7.4.In some embodiments, the pH is 7.4.

In some embodiments, the pH buffer has a pKa between 6.0 and 8.2. Accordingly, in some embodiments, the pH buffer has a pKa of about 6.2, 6.4, 6.6, 6.8, 7.0, 7.2, 7.4, 7.6, 7.8, 8.0, or 8.2. In some embodiments, the pH buffer has a pKa of about 6.2. In some embodiments, the pH buffer has a pKa of about 6.4. In some embodiments, the pH buffer has a pKa of about 6.6. In some embodiments, the pH buffer has a pKa of about 6.8. In some embodiments, the pH buffer has a pKa of about 7.0. In some embodiments, the pH buffer has a pKa of about 7.2. In some embodiments, the pH buffer has a pKa of about 7.4. In some embodiments, the pH buffer has a pKa of about 7.6. In some embodiments, the pH buffer has a pKa of about 7.8. In some embodiments, the pH buffer has a pKa of about 8.0. In some embodiments, the pH buffer has a pKa of about 8.2.

In some embodiments, the buffer is selected from the group consisting of a phosphate buffer, a citrate buffer, an imidazole buffer, a histidine buffer, and a Good's buffer. Accordingly, in some embodiments, the buffer is a phosphate buffer. In some embodiments, the buffer is a citrate buffer. In some embodiments, the buffer is an imidazole buffer. In some embodiments, the buffer is a histidine buffer. In some embodiments, the buffer is a Good's buffer. In some embodiments, the Good's buffer is a Tris buffer or HEPES buffer.

In some embodiments, the pH buffer is a phosphate buffer (e.g., a citrate-phosphate buffer), a Tris buffer, or an imidazole buffer.

In some embodiments, the minimum buffered ionic strength is at least 75 mM, at least 100 mM, at least 125 mM, at least 150 mM, or at least 200 mM. Accordingly, in some embodiments, the minimum buffered ionic strength is at least 75 mM. In some embodiments, the minimum buffered ionic strength is at least 100 mM. In some embodiments, the minimum buffered ionic strength is at least 125 mM. In some embodiments, the minimum buffered ionic strength is at least 150 mM. In some embodiments, the minimum buffered ionic strength is at least 200 mM.

In some embodiments, the minimum buffered ionic strength is between about 75 mM-200 mM, 75 mM-150 mM, 75 mM-100 mM, or 100 mM-200 mM. Accordingly, in some embodiments, the minimum buffered ionic strength is between about 75 mM-200 mM. In some embodiments, the minimum buffered ionic strength is between about 75 mM-150 mM. In some embodiments, the minimum buffered ionic strength is between about 75 mM-100 mM mM. In some embodiments, the minimum buffered ionic strength is between about 100 mM-200 mM.

In some embodiments, the minimum buffered ionic strength is obtained by either increasing buffer concentration in the formulation and/or increasing salt concentration in the formulation. Accordingly, in some embodiments the minimum buffered ionic strength is obtained by increasing buffer concentration. In some embodiments, the minimum buffered ionic strength is obtained by increasing the salt concentration of the formulation. In some embodiments, the minimum buffered ionic strength is obtained by increasing the buffer concentration in the formulation and by increasing the salt concentration in the formulation.

In some embodiments, the disaccharide to buffer ratio is between 0.1-0.9. In some embodiments, the disaccharide to buffer ratio is between 0.1-0.7. In some embodiments, the disaccharide to buffer ratio is between 0.2-0.7. In some embodiments, the disaccharide to buffer ratio is between 0.2-0.5.

In some embodiments, the one or more agents that provides ionic strength comprises a salt. In some embodiments, the salt is selected from the group consisting of NaCl, KCl, and CaCl₂. Accordingly, in some embodiments, the salt is NaCl. In some embodiments, the salt is KCl. In some embodiments, the salt is CaCl₂.

In some embodiments, the total concentration of the one or more additional agents that provides ionic strength is between about 50-500 mM, 100-400 mM, or 200-300 mM. Accordingly, in some embodiments, the total concentration of the one or more agents is between about 50-500 mM. In some embodiments, the total concentration of the one or more agents is between about 100-400 mM. In some embodiments, the total concentration of the one or more agents is between about 200-300 mM. In some embodiments, the total concentration of the one or more agents that provide ionic strength is between about 50-300 mM, 50-150 mM, or 75-125 mM. In some embodiments, the total concentration of the one or more agents that provide ionic strength is between about 50-300 mM. In some embodiments, the total concentration of the one or more agents that provide ionic strength is between about 50-150 mM. In some embodiments, the total concentration of the one or more agents that provide ionic strength is between about 75-125 mM.

In some embodiments, the total concentration of pH buffer is between about 100-300 mM, 200-300 mM, or 250-300 mM. Accordingly, in some embodiments, the total concentration of the pH buffer is between about 100-300 mM. In some embodiments, the total concentration of the pH buffer is between 200-300 mM. In some embodiments, the total concentration of the pH buffer is between 250-300 mM. In some embodiments, the total concentration of the pH buffer is between about 15-250 mM, 30-150 mM, or 40-50 mM. Accordingly, in some embodiments, the total concentration of the pH buffer is between about 15-250 mM. In some embodiments, the total concentration of the pH buffer is between about 30-150 mM. In some embodiments, the total concentration of the pH buffer is between about 40-50 mM.

In some embodiments, the total concentration of the pH buffer and the one or more additional agents that provide ionic strength is selected from about 40 mM Tris buffer and about 75-200 mM NaCl, about 50 mM Tris buffer and about 75 mM-200 mM NaCl, about 100 mM Tris buffer and about 75 mM-200mM NaCl, about 40 mM imidazole and about 75 mM-200 mM NaCl, about 50 mM imidazole and 75 mM-200 mM NaCl, and about 100 mM imidazole and 75 mM-200 mM, about 40 mM phosphate and about 75-200 mM NaCl, about 50 mM phosphate and about 75-200 mM NaCl, and about 100 mM phosphate and 75-200 mM NaCl. Accordingly, in some embodiments, the total concentration of the pH buffer and the one or more additional agents that provide ionic strength is about 40 mM Tris buffer and about 75-200 mM NaCl. In some embodiments, the total concentration of the pH buffer and the one or more additional agents that provide ionic strength is about 50 mM Tris buffer and about 75 mM-200 mM NaCl. In some embodiments, the total concentration of the pH buffer and the one or more additional agents that provide ionic strength is about 100 mM Tris buffer and about 75 mM-200 mM NaCl. In some embodiments, the total concentration of the pH buffer and the one or more additional agents that provide ionic strength is about 40 mM imidazole and about 75 mM-200 mM NaCl. In some embodiments, the total concentration of the pH buffer and the one or more additional agents that provide ionic strength is 50 mM imidazole and 75 mM-200 mM NaCl. In some embodiments, the total concentration of the pH buffer and the one or more additional agents that provide ionic strength is 100 mM imidazole and 75 mM-200 mM NaCl. In some embodiments, the total concentration of the pH buffer and the one or more additional agents that provide ionic strength is about 40 mM imidazole, about 75 mM-200 mM NaCl and 2.5-10% trehalose. In some embodiments, the total concentration of the pH buffer and the one or more additional agents that provide ionic strength is 50 mM imidazole, about 75 mM-200 mM NaCl and 2.5-10% trehalose. In some embodiments, the total concentration of the pH buffer and the one or more additional agents that provide ionic strength is 100 mM imidazole, about 75 mM-200 mM NaCl and 2.5-10% trehalose.

In some embodiments, the ionic strength of the LNP formulation is at least 2.25 times greater than, at least 2.5 times greater than, at least 2.75 times greater than, at least 3 times greater than, at least 3.5 times greater than, at least 4 times greater than, at least 4.5 times greater than, at least 5 times greater than, the minimum buffered ionic strength. Accordingly, in some embodiments, the ionic strength of the LNP formulation is at least 2.25 times greater than the minimum buffered ionic strength. In some embodiments, the ionic strength of the LNP formulation is at least 2.5 times greater than the minimum buffered ionic strength. In some embodiments, the ionic strength of the LNP formulation is at least 2.75 times greater than the minimum buffered ionic strength. In some embodiments, the ionic strength of the LNP formulation is at least 3.0 times greater than the minimum buffered ionic strength. In some embodiments, the ionic strength of the LNP formulation is at least 3.5 times greater than the minimum buffered ionic strength. In some embodiments, the ionic strength of the LNP formulation is at least 4.0 times greater than the minimum buffered ionic strength. In some embodiments, the ionic strength of the LNP formulation is at least 4.5 times greater than the minimum buffered ionic strength. In some embodiments, the ionic strength of the LNP formulation is at least 5.0 times greater than the minimum buffered ionic strength.

In some embodiments, the ionic strength of the LNP formulation is less than 20 times, less than 19 times, less than 18 times, less than 17 times, less than 16 times, less than 15 times, less than 14 times, less than 13 times, less than 12 times, less than 11 times, less than 10 times, less than 9 times, less than 8 times, less than 7 times, less than 6 times, less than 5 times, less than 4 times, the minimum buffered ionic strength. Accordingly, in some embodiments, the ionic strength of the LNP formulation is less than 20 times the minimum buffered ionic strength. In some embodiments, the ionic strength of the LNP formulation is less than 19 times the minimum buffered ionic strength. In some embodiments, the ionic strength of the LNP formulation is less than 18 times the minimum buffered ionic strength. In some embodiments, the ionic strength of the LNP formulation is less than 17 times the minimum buffered ionic strength. In some embodiments, the ionic strength of the LNP formulation is less than 16 times the minimum buffered ionic strength. In some embodiments, the ionic strength of the LNP formulation is less than 15 times the minimum buffered ionic strength. In some embodiments, the ionic strength of the LNP formulation is less than 14 times the minimum buffered ionic strength. In some embodiments, the ionic strength of the LNP formulation is less than 13 times the minimum buffered ionic strength. In some embodiments, the ionic strength of the LNP formulation is less than 12 times the minimum buffered ionic strength. In some embodiments, the ionic strength of the LNP formulation is less than 11 times the minimum buffered ionic strength. In some embodiments, the ionic strength of the LNP formulation is less than 10 times the minimum buffered ionic strength. In some embodiments, the ionic strength of the LNP formulation is less than 9 times the minimum buffered ionic strength. In some embodiments, the ionic strength of the LNP formulation is less than 8 times the minimum buffered ionic strength. In some embodiments, the ionic strength of the LNP formulation is less than 7 times the minimum buffered ionic strength. In some embodiments, the ionic strength of the LNP formulation is less than 6 times the minimum buffered ionic strength. In some embodiments, the ionic strength of the LNP formulation is less than 5 times the minimum buffered ionic strength. In some embodiments, the ionic strength of the LNP formulation is less than 4 times the minimum buffered ionic strength.

In some embodiments, the ionic strength of the LNP formulation is at least two times greater and less than 20 times greater than the minimum buffered ionic strength, and wherein the ionic strength of the LNP formulation is between about 150 mM-750 mM, 150 mM-500 mM, 150 mM-400 mM, 150 mM-300 mM, 150 mM and 200 mM. Accordingly, in some embodiments, the ionic strength of the LNP formulation is at least two times greater and less than 20 times greater than the minimum buffered ionic strength, and wherein the ionic strength of the LNP formulation is between about 150 mM-750 mM. In some embodiments, the ionic strength of the LNP formulation is at least two times greater and less than 20 times greater than the minimum buffered ionic strength, and wherein the ionic strength of the LNP formulation is between about 150 mM-500 mM. In some embodiments, the ionic strength of the LNP formulation is at least two times greater and less than 20 times greater than the minimum buffered ionic strength, and wherein the ionic strength of the LNP formulation is between about 150 mM-400 mM. In some embodiments, the ionic strength of the LNP formulation is at least two times greater and less than 20 times greater than the minimum buffered ionic strength, and wherein the ionic strength of the LNP formulation is between about 150 mM-300 mM. In some embodiments, the ionic strength of the LNP formulation is at least two times greater and less than 20 times greater than the minimum buffered ionic strength, and wherein the ionic strength of the LNP formulation is between about 150 mM and 200 mM.

In some embodiments, the ionic strength of the LNP formulation is at least two times greater and less than 20 times greater than the minimum buffered ionic strength, and wherein the ionic strength of the LNP formulation is or is greater than 150 mM.

In some embodiments, less aggregation is determined by turbidity analysis. In some embodiments, less degradation of the encapsulated mRNA is determined by turbidity analysis. Various ways of measuring turbidity can be used, including for example using visual analysis and/or the use of spectrometry.

In some embodiments, following more than three rounds of freezing at −20° C. and rethawing, the LNP formulation exhibits (i) less aggregation, (ii) less degradation of the encapsulated mRNA, or (iii) both (i) and (ii), as compared to an identical LNP formulation that has only the minimum buffered ionic strength in the LNP formulation instead of an ionic strength at is at least two times greater than the minimum buffered ionic strength.

In some embodiments, the LNPs have a diameter of less than about 100 nm. In some embodiments, the LNPs have a diameter between about 70 nm-90 nm. For example, in some embodiments, the LNPs have a diameter of between about 70 nm-85 nm. In some embodiments, the LNPs have a diameter of between about 70 nm-80 nm. In some embodiments, the LNPs have a diameter of between about 70 nm-75 nm. In some embodiments, the LNPs have a diameter of between about 80 nm-90 nm. In some embodiments, the LNPs have a diameter of between about 85 nm-90 nm. In some embodiments, the LNPs have a diameter of between about 75 nm-90 nm. In some embodiments, the LNPs have a diameter of between about 75 nm-85 nm. In some embodiments, the LNPs have a diameter of between about 75 nm-80 nm. In some embodiments, the LNPs have a diameter of less than about 70 nm.

In some embodiments, the lipid component comprises or consists of DMG-PEG-2000, cKK-E10, cholesterol, and DOPE. Accordingly, in some embodiments, the lipid component comprises DMG-PEG-2000, cKK-E10, cholesterol, and DOPE. In some embodiments, the lipid component consists of DMG-PEG-2000, cKK-E10, cholesterol, and DOPE.

In some embodiments, the N/P ratio is between about 3-5. For example, in some embodiments, the N/P ratio is about 3. In some embodiments, the N/P ratio is about 4. In some embodiments, the N/P ratio is about 5.

In some embodiments, the mRNA is at a final concentration of between about 0.05 mg/mL and 1.0 mg/mL. In some embodiments, the mRNA is at a final concentration of about 0.05 mg/mL. In some embodiments, the mRNA is at a final concentration of about 0.1 mg/mL. In some embodiments, the mRNA is at a final concentration of about 0.1 mg/mL. In some embodiments, the mRNA is at a final concentration of about 0.2 mg/mL. In some embodiments, the mRNA is at a final concentration of about 0.3 mg/mL. In some embodiments, the mRNA is at a final concentration of about 0.4 mg/mL. In some embodiments, the mRNA is at a final concentration of about 0.5 mg/mL. In some embodiments, the mRNA is at a final concentration of about 0.6 mg/mL. In some embodiments, the mRNA is at a final concentration of about 0.7 mg/mL. In some embodiments, the mRNA is at a final concentration of about 0.8 mg/mL. In some embodiments, the mRNA is at a final concentration of about 0.9 mg/mL. In some embodiments, the mRNA is at a final concentration of about 1.0 mg/mL.

In some embodiments, the mRNA is at a concentration of between about 0.2 mg/mL and 0.5 mg/mL.

In some embodiments, the LNPs are stable at −20° C. for at least 3 months, 6 months, 12 months, or more than 12 months. Accordingly, in some embodiments, the LNPs are stable at −20° C. for at least 3 months. In some embodiments, the LNPs are stable at −20° C. for at least 6 months. In some embodiments, the LNPs are stable at −20° C. for at least 12 months. In some embodiments, the LNPs are stable at −20° C. for more than 12 months.

In some embodiments, the LNP formulation is stable following dilution.

In some embodiments, subcutaneous or intramuscular delivery of the formulation is accompanied with reduced pain in comparison to a formulation that does not comprise a buffer having a concentration of or below 300 mM and a pH of between about 7.0 and 7.5.

In some embodiments, the reduced pain is assessed by a 10-cm visual analog scale (VAS) or a six-item verbal rating scale (VRS). Accordingly, in some embodiments, the reduced pain is assessed by a 10-cm visual analog scale (VAS). In some embodiments, the reduced pain is assessed by a six-item verbal rating scale (VRS).

In some aspects, a method of reducing LNP degradation and/or aggregation is provided, the method comprising storing the LNP in the formulation as described herein.

In this application, the use of “or” means “and/or” unless stated otherwise. As used in this disclosure, the term “comprise” and variations of the term, such as “comprising” and “comprises,” are not intended to exclude other additives, components, integers or steps. As used in this application, the terms “about” and “approximately” are used as equivalents. Both terms are meant to cover any normal fluctuations appreciated by one of ordinary skill in the relevant art.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWING

The drawings are for illustration purposes only not for limitation.

FIG. 1A is a graph that shows stability of an LNP at pH 7.5 as a function of increasing the concentration of a trehalose in an LNP formulation and also as a function of the minimum buffer strength needed to maintain LNP stability at pH 7.5. FIG. 1B is a graph that shows stability of an LNP formulation having trehalose at a constant percentage of the LNP formulation (i.e., 2.7%) as a function of fluctuations of pH and as a function of minimum buffer strength needed to maintain LNP formulation stability.

FIG. 2 is a graph that shows lipid pKa dependent behaviour of tested LNP formulations. For these studies, the LNP formulation comprised trehalose at 2.7%.

FIG. 3A depicts various conditions for LNP formulations tested. The table depicts the molar concentration of lipids and the concentration of Tris buffer at pH 7.5. Checkmarks in the table represent LNP formulations that were stable. An “X” represents LNP formulations that were unstable. FIG. 3B is a graph that shows expression of human EPO protein derived from LNPs that encapsulated human EPO mRNA at either 6 hours or 24 hours following administration in an animal model. Various LNP constituent lipids are shown.

FIG. 4A depicts a series of tables that show various compositions of LNP formulations tested. The tables depict the molar concentration of buffers tested (i.e., Tris, or Imidazole) and the corresponding salt concentrations tested (i.e., NaCl) in various LNP formulations assessed. Checkmarks in the table represent LNP formulations that were stable. An “X” represents LNP formulations that were unstable. FIG. 4B depicts a table in which various LNP formulations were assessed. The LNP formulations varied with respect to the concentrations of either Tris or Phosphate buffer. LNP post-dilution stability was assessed. The stable LNPs are indicated with a checkmark, whereas the non-stable LNP formulations are indicated by an “X.”

FIG. 5A depicts a graph of percent encapsulation efficiency of LNP formulations comprising varying trehalose to PBS ratio (e.g. about 0.2-0.5) at 4° C. FIG. 5B depicts a graph of percent encapsulation efficiency of LNP formulations comprising varying trehalose to PBS ratio (e.g. about 0.2-0.5) at 25° C.

FIG. 6A depicts a graph of LNP sizes (in nanometers) of LNP formulations comprising varying trehalose to PBS ratio (e.g. about 0.2-0.5) at 4° C. FIG. 6B depicts a graph of LNP sizes (in nanometers) of LNP formulations comprising varying trehalose to PBS ratio (e.g. about 0.2-0.5) at 25° C.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “batch” refers to a quantity or amount of mRNA synthesized at one time, e.g., produced according to a single manufacturing order during the same cycle of manufacture. A batch may refer to an amount of mRNA synthesized in one reaction that occurs via a single aliquot of enzyme and/or a single aliquot of DNA template for continuous synthesis under one set of conditions. In some embodiments, a batch would include the mRNA produced from a reaction in which not all reagents and/or components are supplemented and/or replenished as the reaction progresses. The term “not in a single batch” would not mean mRNA synthesized at different times that are combined to achieve the desired amount.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery). In some embodiments, delivery is pulmonary delivery, e.g., comprising nebulization.

Encapsulation: As used herein, the term “encapsulation,” or grammatical equivalent, refers to the process of confining an mRNA molecule within a nanoparticle.

Engineered or mutant: As used herein, the terms “engineered” or “ mutant”, or grammatical equivalents refer to a nucleotide or protein sequence comprising one or more modifications compared to its naturally-occurring sequence, including but not limited to deletions, insertions of heterologous nucleic acids or amino acids, inversions, substitutions, or combinations thereof.

Expression: As used herein, “expression” of a nucleic acid sequence refers to translation of an mRNA into a polypeptide, assemble multiple polypeptides (e.g., heavy chain or light chain of antibody) into an intact protein (e.g., antibody) and/or post-translational modification of a polypeptide or fully assembled protein (e.g., antibody). In this application, the terms “expression” and “production,” and grammatical equivalents, are used interchangeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Impurities: As used herein, the term “impurities” refers to substances inside a confined amount of liquid, gas, or solid, which differ from the chemical composition of the target material or compound. Impurities are also referred to as contaminants.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

messenger RNA (mRNA): As used herein, the term “messenger RNA (mRNA)” refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery. In some embodiments, the nucleotides T and U are used interchangeably in sequence descriptions.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Stable: As used herein, the term “stable” protein or its grammatical equivalents refer to protein that retains its physical stability and/or biological activity. In one embodiment, protein stability is determined based on the percentage of monomer protein in the solution, at a low percentage of degraded (e.g., fragmented) and/or aggregated protein. In one embodiment, a stable engineered protein retains or exhibits an enhanced half-life as compared to a wild-type protein. In one embodiment, a stable engineered protein is less prone to ubiquitination that leads to proteolysis as compared to a wild-type protein.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present invention provides, among other things, improved methods and compositions that result in the production of stable LNP formulations encapsulating mRNA which are resistant to multiple freeze/thaw cycles. Such resistance to multiple freeze/thaw cycles is manifested at least by 1) low aggregation of the LNPs following one or more freeze/thaw cycles; and 2) low degradation of the encapsulated mRNA.

### Stable Lipid Nanoparticle Formulations

Provided herein are formulations for stable liquid lipid nanoparticles (LNP) encapsulating mRNA encoding a peptide or polypeptide. Such stable LNPs are resistant to aggregation and to mRNA degradation following one or more freeze thaw cycles. For example, the stable LNPs are resistant to one, two, three, four, five or more than 5 freeze thaw cycles, where the LNP encapsulating mRNAs are stored at −20° C. In some embodiments, the stable LNPs are resistant to one, two, three, four, five or more than 5 freeze thaw cycles, where the LNP encapsulating mRNAs are stored at −80° C. or below.

Furthermore, the stable LNP encapsulating mRNA formulations described herein are accompanied by reduced pain when administered to a subject in need thereof. For example, the described LNP formulations result in reduced pain upon administration, such as by intramuscular or subcutaneous administration, in comparison to LNP formulations that do not have certain ionic strengths as those described herein.

In some embodiments, such stable LNP formulations comprise: a) one or more LNPs having a lipid component comprising a cationic lipid, a non-cationic lipid, a PEG-modified lipid and optionally cholesterol; b) mRNA encapsulated within the one or more lipid nanoparticles and encoding a peptide or polypeptide; c) a sugar or a sugar alcohol; d) an LNP formulation pH of from 6.0 to 8.0; e) a pH buffer that at a minimum buffered ionic strength provides the LNP formulation pH; and optionally f) one or more additional agents that provide ionic strength to the LNP formulation. The stable LNP formulations have a total concentration of pH buffer from (e), and optionally one or more additional agents from (f), that provide(s) an ionic strength of the LNP formulation that is at least two times greater than the minimum buffered ionic strength. Following one, two, three rounds or more than three rounds of freezing and thawing, the LNP formulations described has (i) less aggregation, (ii) less degradation of the encapsulated mRNA, or (iii) both (i) and (ii), as compared to an identical LNP formulation that has only the minimum buffered ionic strength in the LNP formulation instead of an ionic strength that is at least two times greater than the minimum buffered ionic strength.

The one or more additional agents in (f) above can be a salt, a buffer or a combination of a salt and a buffer. For example, the one or more additional agents in (f), can include for example NaCl, KCl, and CaCl₂. The buffer includes, for example, a phosphate buffer, a citrate buffer, an imidazole buffer, a histidine buffer, or a Good's buffer. Various kinds of Good's buffer are known the art, and include, for example, MES, Bis-tris methane, ADA, Bis-tris propane, PIPES, ACES, POPSO, Cholamine chloride, MOPS, BES, AMPB, TES, HEPES, DIPSO, MOBS, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, CAPSO, AMP, CAPS, and CABS. In some embodiments, the Good's buffer is either a Tris buffer or a HEPES buffer.

In some embodiments, the one or more additional agents have a concentration of between about 50-500 mM, 100-400 mM, or 200-300 mM. The buffer pH of the LNP formulations described herein have a concentration of between about 100-300 mM, 200-300 mM, or 250-300 mM.

The minimum buffered ionic strength of the stable LNP formulation encapsulating mRNA as described herein is, for example, at least 15 mM, at least 25 mM, at least 50 mM, at least 75 mM, at least 100 mM, at least 125 mM, at least 150 mM, or at least 200 mM. In embodiments, the stable LNP formulation encapsulating mRNA as described herein, is for example, between about 15 mM-200 mM, 50 mM-200 mM, 75 mM-200 mM, 15 mM-150 mM, 50 mM-150 mM, 75 mM-150 mM, 15 mM-100 mM, 50 mM-100 mM, 75 mM-100 mM, or 100 mM-200 mM. The minimum buffered ionic strength can be obtained in various ways. For example, in some embodiments, the minimum buffered ionic strength is obtained by increasing the buffer concentration. Alternatively, the minimum buffered ionic strength is obtained by increasing the salt concentration. In some embodiments, the minimum buffered ionic strength is obtained by increasing both the buffer concentration and the salt concentration. For example, in some embodiments, the total concentration of the pH buffer and the one or more additional agents that provide ionic strength is selected from about 40 mM Tris buffer and about 75-200 mM NaCl, about 50 mM Tris buffer and about 75 mM-200 mM NaCl, about 100 mM Tris buffer and about 75 mM-200 mM NaCl, about 40 mM imidazole and about 75 mM-200 mM NaCl, about 50 mM imidazole and 75 mM-200 mM NaCl and about 100 mM imidazole and 75 mM-200 mM NaCl, about 40 mM phosphate and about 75 mM-200 mM NaCl, about 50 mM phosphate and 75 mM-200 mM NaCl and about 100 mM phosphate and 75 mM-200 mM NaCl. In some embodiments, the total concentration of the pH buffer and the one or more additional agents that provide ionic strength is selected from 40 mM Tris buffer, about 75-200 mM NaCl, and about 2.5-10% trehalose, about 50 mM Tris buffer, about 75 mM-200 mM NaCl and about 2.5-10% trehalose, about 100 mM Tris buffer, about 75 mM-200 mM NaCl and about 2.5-10% trehalose, about 40 mM imidazole, about 75 mM-200 mM NaCl and about 2.5-10% trehalose, about 50 mM imidazole, 75 mM-200 mM NaCl and about 2.5-10% trehalose, and about 100 mM imidazole, 75 mM-200 mM NaCl and about 2.5-10% trehalose, about 40 mM phosphate, about 75 mM-200 mM NaCl and about 2.5-10% trehalose, about 50 mM phosphate, 75 mM-200 mM NaCl and about 2.5-10% trehalose, about 100 mM phosphate 75 mM-200 mM NaCl and about 2.5-10% trehalose.

In some embodiments, the buffers are used interchangeably. In some embodiments, the Tris buffer is substituted with an imidazole buffer or a phosphate buffer. In some embodiments, the Tris buffer is substituted with an imidazole buffer. In some embodiments, the Tris buffer is substituted with a phosphate buffer. In some embodiments, the imidazole buffer is substituted with a phosphate buffer or a Tris buffer. In some embodiments, the imidazole buffer is substituted with a phosphate buffer. In some embodiments, the imidazole buffer is substituted with a Tris buffer. In some embodiments, the phosphate buffer is substituted with a Tris buffer or an imidazole buffer. In some embodiments, the phosphate buffer is substituted with a Tris buffer. In some embodiments, the phosphate buffer is substituted with an imidazole buffer.

In some embodiments, the Tris buffer, imidazole buffer or phosphate buffer have a high buffer strength (e.g., 100 mM or greater). In some embodiments, the Tris buffer, phosphate buffer or imidazole buffer at a low buffer strength (e.g., 15-20 mM) is used with a high salt concentration (e.g., 200 mM or greater NaCl). In some embodiments, the Tris buffer, phosphate buffer or imidazole buffer at a medium buffer strength (e.g., 40-50 mM) is used with a medium salt concentration (e.g., 50-100 mM NaCl).

In some embodiments, the Tris buffer, phosphate buffer or imidazole buffer is used with a low trehalose concentration (e.g., 50-100 mM NaCl). In some embodiments, LNP formulation stability was greater at low sugar to buffer ratio. In some embodiments, the lower trehalose to buffer ratio of the LNP formulation was beneficial in preventing a decrease in encapsulation. In some embodiments, the lower trehalose to buffer ratio prevented an increase in LNP size.

In some embodiments, the LNP formulations have an ionic strength that is at least 2.25 times greater than, at least 2.5 times greater than, at least 2.75 times greater than, at least 3 times greater than, at least 3.5 times greater than, at least 4 times greater than, at least 4.5 times greater than, at least 5 times greater than, the minimum buffered ionic strength. In some embodiments, the LNP formulations have an ionic strength that is less than 20 times, less than 19 times, less than 18 times, less than 17 times, less than 16 times, less than 15 times, less than 14 times, less than 13 times, less than 12 times, less than 11 times, less than 10 times, less than 9 times, less than 8 times, less than 7 times, less than 6 times, less than 5 times, less than 4 times, the minimum buffered ionic strength. In some embodiments, the ionic strength of the LNP formulation is at least two times greater and less than 20 times greater than the minimum buffered ionic strength, and wherein the ionic strength of the LNP formulation is between about 150 mM-750 mM, 150 mM-500 mM, 150 mM-400 mM, 150 mM-300 mM, 150 mM and 200 mM. In some embodiments, the ionic strength of the LNP formulation is at least two times greater and less than 20 times greater than the minimum buffered ionic strength, and wherein the ionic strength of the LNP formulation is or is greater than 150 mM. The minimum buffered ionic strength referenced throughout is at least 75 mM, at least 100 mM, at least 125 mM, at least 150 mM, or at least 200 mM.

In some embodiments, the stable LNP formulations described herein further comprise one or cryoprotectants. Cryoprotectants can be characterized as either “penetrating” cryoprotectants or “non-penetrating” cryoprotectants. Suitable cryoprotectants for the LNP formulations described herein can be selected from penetrating cryoprotectants and/or non-penetrating cryoprotectants. Exemplary non-penetrating cryoprotectants include, for example, sugars, such as dextrose, sorbitol, trehalose, sucrose, raffinose, dextran, and inulin. Another category of non-penetrating cryoprotectants include, for example polymers, such as PVP, PVA, Poloxamer, and PEG. Exemplary penetrating cryoprotectants include, for example, glycerol, ethylene glycol, tri-ethylene glycol, propylene glycol, tetra-ethylene glycol. Any one or more of the described cryoprotectants are suitable for inclusion in the stable LNP formulations described herein. In some embodiments, the cryoprotectant in the LNP formulation comprises trehalose at a concentration between 1% and 20%. In some embodiments, the cryoprotectant in the LNP formulation comprises trehalose at a concentration of between about 2.5%-3.0%. In some embodiments, the cryoprotectants in the LNP formulation comprises trehalose at a concentration of about 2.5%. In some embodiments, the cryoprotectants in the LNP formulation comprises trehalose at a concentration of about 2.6%. In some embodiments, the cryoprotectants in the LNP formulation comprises trehalose at a concentration of about 2.7%. In some embodiments, the cryoprotectants in the LNP formulation comprises trehalose at a concentration of about 2.8%. In some embodiments, the cryoprotectants in the LNP formulation comprises trehalose at a concentration of about 2.9%. In some embodiments, the cryoprotectants in the LNP formulation comprises trehalose at a concentration of about 3.0%.

Various non-cationic lipids can be used in the LNP formulation described herein. For example, a suitable cationic lipid for the LNP formulation describe herein can be selected from 1,2-Dierucoyl-sn-glycero-3-phosphoethanolamine (DEPE), di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, or 1-stearoyl-2-oleoyl-phosphatidyethanolamine (SOPE). In some embodiments, the non-cationic lipid is DOPE.

The non-cationic lipid in the LNP formulation can be at a lipid molar ratio greater than 10%. For example, in some embodiments, the non-cationic lipid is at a lipid molar ratio of 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%.

In some embodiments, the cationic lipid is selected from a lipidoid. Various lipidoids are known in the art. For example, lipidoids are described in Goldberg M. (2013) Lipidoids: A Combinatorial Approach to siRNA Delivery. In: Howard K (eds) RNA Interference from Biology to Therapeutics. Advances in Delivery Science and Technology. Springer, Boston, Mass., the contents of which are incorporated herein by reference. In some embodiments, the lipidoid is cationic. In some embodiments, the lipidoid contains up to seven tails. The seven tails can emanate, for example, from the amine backbone. In some embodiments, the lipidoid has an inversion of its ester linkage with respect to an aliphatic chain when compared to natural lipids such as triglycerides. In some embodiments, the lipidoid does not have an inversion of its ester linkage with respect to an aliphatic chain when compared to natural lipids such as triglycerides.

In some embodiments, the lipidoid includes for example aminoalcohol lipidoids. In some embodiments, the lipidoid is selected from cKK-E10, OF-02, or C12-200. Accordingly, in some embodiments, the lipidoid is cKK-E-10. In some embodiments, the lipidoid is OF-02. In some embodiments, the lipidoid is C12-200.

The LNP formulations of the present invention can have a pH between about 6.0 and 8.0. For example, in some embodiments, the LNP formulations can have a pH of between about 6.0-7.0. In some embodiments, the LNP formulations can have a pH of between about 6.5-7.5. In some embodiments, the LNP formulations can have a pH of between about 7.0-8.0. In some embodiments, the LNP formulation has a pH of about 7.4. In some embodiments, the LNP formulation has a pH that is equivalent to physiological pH.

The pH buffer of LNP formulations can have a pKa between about 6.0 and 8.2. For example, the pH buffer of the LNP formulations has a pKa of about 6.2, 6.4, 6.6, 6.8, 7.0, 7.2, 7.4, 7.6, 7.8, 8.0, or 8.2. In some embodiments, the pH buffer has a pKa of about 6.2. In some embodiments, the pH buffer has a pKa of about 6.4. In some embodiments, the pH buffer has a pKa of about 6.6. In some embodiments, the pH buffer has a pKa of about 6.8. In some embodiments, the pH buffer has a pKa of about 7.0. In some embodiments, the pH buffer has a pKa of about 7.2. In some embodiments, the pH buffer has a pKa of about 7.4. In some embodiments, the pH buffer has a pKa of about 7.6. In some embodiments, the pH buffer has a pKa of about 7.8. In some embodiments, the pH buffer has a pKa of about 8.0. In some embodiments, the pH buffer has a pKa of about 8.2.

As described above, the LNP formulations described herein have less aggregation following one or more freeze thaw cycles. There are various ways in the art to determine LNP aggregation, including for example, determined by any one of dynamic light scattering (DLS), nanoparticle tracking analysis (NTA), turbidity analysis, flow microscopy analysis, flow cytometry, FTIR microscopy, resonant mass measurement (RMM), Raman microscopy, filtration, laser diffraction, electron microscopy, atomic force microscopy (AFM), static light scattering (SLS), multi-angle static light scattering (MALS), field flow fractionation (FFF), or analytical ultracentrifugation (AUC). Any one or more of these methods can be used to assess LNP aggregation.

The LNP formulations described herein also have less mRNA degradation following one of more freeze thaw cycles. There are various ways in the art to determine mRNA degradation, such as for example, dynamic light scattering (DLS), nanoparticle tracking analysis (NTA), turbidity analysis, flow microscopy analysis, flow cytometry, FTIR microscopy, resonant mass measurement (RMM), Raman microscopy, filtration, laser diffraction, electron microscopy, atomic force microscopy (AFM), static light scattering (SLS), multi-angle static light scattering (MALS), field flow fractionation (FFF), and analytical ultracentrifugation (AUC). Any one or more of these methods can be used to assess mRNA degradation.

The LNP formulations described herein have a diameter of less than 100 nm. For example, in some embodiments, the LNPs have a diameter between 70 nm-90 nm. In some embodiments, the LNPs have a diameter of less than 70 nm.

As described throughout, various kinds of lipid components are suitable for the LNPs described herein. In some embodiments, the LNP formulation has a lipid component that comprises DMG-PEG-2000, cKK-E10, cholesterol, and DOPE. In some embodiments, the LNP formulation has a lipid component that consists of DMG-PEG-2000, cKK-E10, cholesterol, and DOPE.

The LNP formulations can have a range of N/P ratio from about 3-5. In some embodiments, the N/P ratio is about 3. In some embodiments, the N/P ratio is about 4. In some embodiments, the N/P ratio is about 5.

The LNP formulations encapsulate mRNA. Any mRNA can be encapsulated by the LNP formulations described herein. The final concentration of mRNA encapsulated within the LNP can range from between about 0.05 mg/mL and 1.0 mg/mL. In some embodiments, the mRNA encapsulated within the LNP ranges from about 0.2 mg/mL to about 0.5 mg/mL.

The LNP formulations described herein are stable when stored at −20° C., −80° C., or less than −80° C. Accordingly, in some embodiments, the LNP formulations described herein are stable when stored at −20° C. In some embodiments, the LNP formulations described herein are stable when stored at −80° C. In some embodiments, the LNP formulations described herein are stable when stored at below −80° C. For example, the LNP formulations are stable for at least 3 months, 6 months, 12 months, or more than 12 months when stored at −20° C. Furthermore, the LNP formulations are stable following dilution.

Synthesis of mRNA

mRNAs according to the present invention may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

In some embodiments, for the preparation of mRNA according to the invention, a DNA template is transcribed in vitro. A suitable DNA template typically has a promoter, for example a T3, T7 or SP6 promoter, for in vitro transcription, followed by desired nucleotide sequence for desired mRNA and a termination signal.

Synthesis of mRNA using SP6 RNA Polymerase

In some embodiments, mRNA is produced using SP6 RNA Polymerase. SP6 RNA Polymerase is a DNA-dependent RNA polymerase with high sequence specificity for SP6 promoter sequences. The SP6 polymerase catalyzes the 5′→3′ in vitro synthesis of RNA on either single-stranded DNA or double-stranded DNA downstream from its promoter; it incorporates native ribonucleotides and/or modified ribonucleotides and/or labeled ribonucleotides into the polymerized transcript. Examples of such labeled ribonucleotides include biotin-, fluorescein-, digoxigenin-, aminoallyl-, and isotope-labeled nucleotides.

The sequence for bacteriophage SP6 RNA polymerase was initially described (GenBank: Y00105.1) as having the following amino acid sequence:

MQDLHAIQLQLEEEMFNGGIRRFEADQQRQIAAGSESDTAWNRRLLSELI
APMAEGIQAYKEEYEGKKGRAPRALAFLQCVENEVAAYITMKVVMDMLNT
DATLQAIAMSVAERIEDQVRFSKLEGHAAKYFEKVKKSLKASRTKSYRHA
HNVAVVAEKSVAEKDADFDRWEAWPKETQLQIGTTLLEILEGSVFYNGEP
VFMRAMRTYGGKTIYYLQTSESVGQWISAFKEHVAQLSPAYAPCVIPPRP
WRTPFNGGFHTEKVASRIRLVKGNREHVRKLTQKQMPKVYKAINALQNTQ
WQINKDVLAVIEEVIRLDLGYGVPSFKPLIDKENKPANPVPVEFQHLRGR
ELKEMLSPEQWQQFINWKGECARLYTAETKRGSKSAAVVRMVGQARKYSA
FESIYFVYAMDSRSRVYVQSSTLSPQSNDLGKALLRFTEGRPVNGVEALK
WFCINGANLWGWDKKTFDVRVSNVLDEEFQDMCRDIAADPLTFTQWAKAD
APYEFLAWCFEYAQYLDLVDEGRADEFRTHLPVHQDGSCSGIQHYSAMLR
DEVGAKAVNLKPSDAPQDIYGAVAQVVIKKNALYMDADDATTFTSGSVTL
SGTELRAMASAWDSIGITRSLTKKPVMTLPYGSTRLTCRESVIDYIVDLE
EKEAQKAVAEGRTANKVHPFEDDRQDYLTPGAAYNYMTALIWPSISEVVK
APIVAMKMIRQLARFAAKRNEGLMYTLPTGFILEQKIMATEMLRVRTCLM
GDIKMSLQVETDIVDEAAMMGAAAPNFVHGHDASHLILTVCELVDKGVTS
IAVIHDSFGTHADNTLTLRVALKGQMVAMYIDGNALQKLLEEHEVRWMVD
TGIEVPEQGEFDLNEIMDSEYVFA.

An SP6 RNA polymerase suitable for the present invention can be any enzyme having substantially the same polymerase activity as bacteriophage SP6 RNA polymerase. Thus, in some embodiments, an SP6 RNA polymerase suitable for the present invention may be modified from SEQ ID NO: 16. For example, a suitable SP6 RNA polymerase may contain one or more amino acid substitutions, deletions, or additions. In some embodiments, a suitable SP6 RNA polymerase has an amino acid sequence about 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, or 60% identical or homologous to SEQ ID NO: 16. In some embodiments, a suitable SP6 RNA polymerase may be a truncated protein (from N-terminus, C-terminus, or internally) but retain the polymerase activity. In some embodiments, a suitable SP6 RNA polymerase is a fusion protein.

An SP6 RNA polymerase suitable for the invention may be a commercially-available product, e.g., from Aldevron, Ambion, New England Biolabs (NEB), Promega, and Roche. The SP6 may be ordered and/or custom designed from a commercial source or a non-commercial source according to the amino acid sequence of SEQ ID NO: 16 or a variant of SEQ ID NO: 16 as described herein. The SP6 may be a standard-fidelity polymerase or may be a high-fidelity/high-efficiency/high-capacity which has been modified to promote RNA polymerase activities, e.g., mutations in the SP6 RNA polymerase gene or post-translational modifications of the SP6 RNA polymerase itself. Examples of such modified SP6 include SP6 RNA Polymerase-Plus™ from Ambion, HiScribe SP6 from NEB, and RiboMAX™ and Riboprobe® Systems from Promega.

In some embodiments, a suitable SP6 RNA polymerase is a fusion protein. For example, an SP6 RNA polymerase may include one or more tags to promote isolation, purification, or solubility of the enzyme. A suitable tag may be located at the N-terminus, C-terminus, and/or internally. Non-limiting examples of a suitable tag include Calmodulin-binding protein (CBP); Fasciola hepatica 8-kDa antigen (Fh8); FLAG tag peptide; glutathione-S-transferase (GST); Histidine tag (e.g., hexahistidine tag (His6)); maltose-binding protein (MBP); N-utilization substance (NusA); small ubiquitin related modifier (SUMO) fusion tag; Streptavidin binding peptide (STREP); Tandem affinity purification (TAP); and thioredoxin (TrxA). Other tags may be used in the present invention. These and other fusion tags have been described, e.g., Costa et al. Frontiers in Microbiology 5 (2014): 63 and in PCT/US16/57044, the contents of which are incorporated herein by reference in their entireties. In certain embodiments, a His tag is located at SP6′s N-terminus.

DNA Template

Typically, a DNA template is either entirely double-stranded or mostly single-stranded with a double-stranded SP6 promoter sequence.

Linearized plasmid DNA (linearized via one or more restriction enzymes), linearized genomic DNA fragments (via restriction enzyme and/or physical means), PCR products, and/or synthetic DNA oligonucleotides can be used as templates for in vitro transcription with SP6, provided that they contain a double-stranded SP6 promoter upstream (and in the correct orientation) of the DNA sequence to be transcribed.

In some embodiments, the linearized DNA template has a blunt-end.

In some embodiments, the DNA sequence to be transcribed may be optimized to facilitate more efficient transcription and/or translation. For example, the DNA sequence may be optimized regarding cis-regulatory elements (e.g., TATA box, termination signals, and protein binding sites), artificial recombination sites, chi sites, CpG dinucleotide content, negative CpG islands, GC content, polymerase slippage sites, and/or other elements relevant to transcription; the DNA sequence may be optimized regarding cryptic splice sites, mRNA secondary structure, stable free energy of mRNA, repetitive sequences, RNA instability motif, and/or other elements relevant to mRNA processing and stability; the DNA sequence may be optimized regarding codon usage bias, codon adaptability, internal chi sites, ribosomal binding sites (e.g., IRES), premature polyA sites, Shine-Dalgarno (SD) sequences, and/or other elements relevant to translation; and/or the DNA sequence may be optimized regarding codon context, codon-anticodon interaction, translational pause sites, and/or other elements relevant to protein folding. Optimization methods known in the art may be used in the present invention, e.g., GeneOptimizer by ThermoFisher and OptimumGene™, which are described in US 20110081708, the contents of which are incorporated herein by reference in its entirety.

In some embodiments, the DNA template includes a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

Exemplary 3′ and/or 5′ UTR sequences can be derived from mRNA molecules which are stable (e.g., globin, actin, GAPDH, tubulin, histone, or citric acid cycle enzymes) to increase the stability of the sense mRNA molecule. For example, a 5′ UTR sequence may include a partial sequence of a CMV immediate-early 1 (IE1) gene, or a fragment thereof to improve the nuclease resistance and/or improve the half-life of the polynucleotide. Also contemplated is the inclusion of a sequence encoding human growth hormone (hGH), or a fragment thereof to the 3′ end or untranslated region of the polynucleotide (e.g., mRNA) to further stabilize the polynucleotide. Generally, these modifications improve the stability and/or pharmacokinetic properties (e.g., half-life) of the polynucleotide relative to their unmodified counterparts, and include, for example modifications made to improve such polynucleotides' resistance to in vivo nuclease digestion.

Large-Scale mRNA Synthesis

In some embodiments, the present invention can be used in large-scale production of stable LNP encapsulated mRNA. In some embodiments, a method according to the invention synthesizes mRNA at least 100 mg, 150 mg, 200 mg, 300 mg, 400 mg, 500 mg, 600 mg, 700 mg, 800 mg, 900 mg, 1 g, 5 g, 10 g, 25 g, 50 g, 75 g, 100 g, 250 g, 500 g, 750 g, 1 kg, 5 kg, 10 kg, 50 kg, 100 kg, 1000 kg, or more at a single batch. As used herein, the term “batch” refers to a quantity or amount of mRNA synthesized at one time, e.g., produced according to a single manufacturing setting. A batch may refer to an amount of mRNA synthesized in one reaction that occurs via a single aliquot of enzyme and/or a single aliquot of DNA template for continuous synthesis under one set of conditions. mRNA synthesized at a single batch would not include mRNA synthesized at different times that are combined to achieve the desired amount. Generally, a reaction mixture includes SP6 RNA polymerase, a linear DNA template, and an RNA polymerase reaction buffer (which may include ribonucleotides or may require addition of ribonucleotides).

According to the present invention, 1-100 mg of SP6 polymerase is typically used per gram (g) of mRNA produced. In some embodiments, about 1-90 mg, 1-80 mg, 1-60 mg, 1-50 mg, 1-40 mg, 10-100 mg, 10-80 mg, 10-60 mg, 10-50 mg of SP6 polymerase is used per gram of mRNA produced. In some embodiments, about 5-20 mg of SP6 polymerase is used to produce about 1 gram of mRNA. In some embodiments, about 0.5 to 2 grams of SP6 polymerase is used to produce about 100 grams of mRNA. In some embodiments, about 5 to 20 grams of SP6 polymerase is used to about 1 kilogram of mRNA. In some embodiments, at least 5 mg of SP6 polymerase is used to produce at least 1 gram of mRNA. In some embodiments, at least 500 mg of SP6 polymerase is used to produce at least 100 grams of mRNA. In some embodiments, at least 5 grams of SP6 polymerase is used to produce at least 1 kilogram of mRNA. In some embodiments, about 10 mg, 20 mg, 30 mg, 40 mg, 50 mg, 60 mg, 70 mg, 80 mg, 90 mg, or 100 mg of plasmid DNA is used per gram of mRNA produced. In some embodiments, about 10-30 mg of plasmid DNA is used to produce about 1 gram of mRNA. In some embodiments, about 1 to 3 grams of plasmid DNA is used to produce about 100 grams of mRNA. In some embodiments, about 10 to 30 grams of plasmid DNA is used to about 1 kilogram of mRNA. In some embodiments, at least 10 mg of plasmid DNA is used to produce at least 1 gram of mRNA. In some embodiments, at least 1 gram of plasmid DNA is used to produce at least 100 grams of mRNA. In some embodiments, at least 10 grams of plasmid DNA is used to produce at least 1 kilogram of mRNA.

In some embodiments, the concentration of the SP6 RNA polymerase in the reaction mixture may be from about 1 to 100 nM, 1 to 90 nM, 1 to 80 nM, 1 to 70 nM, 1 to 60 nM, 1 to 50 nM, 1 to 40 nM, 1 to 30 nM, 1 to 20 nM, or about 1 to 10 nM. In certain embodiments, the concentration of the SP6 RNA polymerase is from about 10 to 50 nM, 20 to 50 nM, or 30 to 50 nM. A concentration of 100 to 10000 Units/ml of the SP6 RNA polymerase may be used, as examples, concentrations of 100 to 9000 Units/ml, 100 to 8000 Units/ml, 100 to 7000 Units/ml, 100 to 6000 Units/ml, 100 to 5000 Units/ml, 100 to 1000 Units/ml, 200 to 2000 Units/ml, 500 to 1000 Units/ml, 500 to 2000 Units/ml, 500 to 3000 Units/ml, 500 to 4000 Units/ml, 500 to 5000 Units/ml, 500 to 6000 Units/ml, 1000 to 7500 Units/ml, and 2500 to 5000 Units/ml may be used.

The concentration of each ribonucleotide (e.g., ATP, UTP, GTP, and CTP) in a reaction mixture is between about 0.1 mM and about 10 mM, e.g., between about 1 mM and about 10 mM, between about 2 mM and about 10 mM, between about 3 mM and about 10 mM, between about 1 mM and about 8 mM, between about 1 mM and about 6 mM, between about 3 mM and about 10 mM, between about 3 mM and about 8 mM, between about 3 mM and about 6 mM, between about 4 mM and about 5 mM. In some embodiments, each ribonucleotide is at about 5 mM in a reaction mixture. In some embodiments, the total concentration of rNTPs (for example, ATP, GTP, CTP and UTPs combined) used in the reaction range between 1 mM and 40 mM. In some embodiments, the total concentration of rNTPs (for example, ATP, GTP, CTP and UTPs combined) used in the reaction range between 1 mM and 30 mM, or between 1 mM and 28 mM, or between 1 mM to 25 mM, or between 1 mM and 20 mM. In some embodiments, the total rNTPs concentration is less than 30 mM. In some embodiments, the total rNTPs concentration is less than 25 mM. In some embodiments, the total rNTPs concentration is less than 20 mM. In some embodiments, the total rNTPs concentration is less than 15 mM. In some embodiments, the total rNTPs concentration is less than 10 mM.

The RNA polymerase reaction buffer typically includes a salt/buffering agent, e.g., Tris, HEPES, ammonium sulfate, sodium bicarbonate, sodium citrate, sodium acetate, potassium phosphate sodium phosphate, sodium chloride, and magnesium chloride.

The pH of the reaction mixture may be between about 6 to 8.5, about 6.5 to 8.0, about 7.0 to 7.5, and in some embodiments, the pH is 7.5.

Linear or linearized DNA template (e.g., as described above and in an amount/concentration sufficient to provide a desired amount of RNA), the RNA polymerase reaction buffer, and SP6 RNA polymerase are combined to form the reaction mixture. The reaction mixture is incubated at between about 37° C. and about 42° C. for thirty minutes to six hours, e.g., about sixty to about ninety minutes.

In some embodiments, about 5 mM NTPs, about 0.05 mg/mL SP6 polymerase, and about 0.1 mg/ml DNA template in a suitable RNA polymerase reaction buffer (final reaction mixture pH of about 7.5) is incubated at about 37° C. to about 42° C. for sixty to ninety minutes.

In some embodiments, a reaction mixture contains linearized double stranded DNA template with an SP6 polymerase-specific promoter, SP6 RNA polymerase, RNase inhibitor, pyrophosphatase, 29 mM NTPs, 10 mM DTT and a reaction buffer (when at 10× is 800 mM HEPES, 20 mM spermidine, 250 mM MgCl₂, pH 7.7) and quantity sufficient (QS) to a desired reaction volume with RNase-free water; this reaction mixture is then incubated at 37° C. for 60 minutes. The polymerase reaction is then quenched by addition of DNase I and a DNase I buffer (when at 10× is 100 mM Tris-HCl, 5 mM MgCl₂ and 25 mM CaCl₂, pH 7.6) to facilitate digestion of the double-stranded DNA template in preparation for purification. This embodiment has been shown to be sufficient to produce 100 grams of mRNA.

In some embodiments, a reaction mixture includes NTPs at a concentration ranging from 1-10 mM, DNA template at a concentration ranging from 0.01-0.5 mg/ml, and SP6 RNA polymerase at a concentration ranging from 0.01-0.1 mg/ml, e.g., the reaction mixture comprises NTPs at a concentration of 5 mM, the DNA template at a concentration of 0.1 mg/ml, and the SP6 RNA polymerase at a concentration of 0.05 mg/ml.

Nucleotides

Various naturally-occurring or modified nucleosides may be used to product mRNA according to the present invention. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methyl-cytidine (“5 mC”), pseudouridine (“ψU”), and/or 2-thio-uridine (“2sU”). See, e.g., U.S. Pat. No. 8,278,036 or WO2011012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US20120195936 and international publication WO2011012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

Post-Synthesis Processing

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published U.S. Patent Application Publication No. 2016/0032356 and U.S. Provisional Patent Application No. 62/464,327, filed Feb. 27, 2017, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly A or poly C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly (A) and poly (C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

mRNA synthesized according to the present invention may be used without further purification. In particular, mRNA synthesized according to the present invention may be used without a step of removing shortmers. In some embodiments, mRNA synthesized according to the present invention may be further purified. Various methods may be used to purify mRNA synthesized according to the present invention. For example, purification of mRNA can be performed using centrifugation, filtration and/or chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol: chloroform: isoamyl alcohol solution, well known to one of skill in the art. In some embodiments, the mRNA is purified using Tangential Flow Filtration. Suitable purification methods include those described in U.S. Patent Application Publication No. 2016/0040154, U.S. Patent Application Publication No. 2015/0376220, International Patent Application PCT/US18/19954 entitled “METHODS FOR PURIFICATION OF MESSENGER RNA” filed on Feb. 27, 2018, and International Patent Application PCT/US18/19978 entitled “METHODS FOR PURIFICATION OF MESSENGER RNA” filed on Feb. 27, 2018, all of which are incorporated by reference herein and may be used to practice the present invention.

In some embodiments, the mRNA is purified before capping and tailing. In some embodiments, the mRNA is purified after capping and tailing. In some embodiments, the mRNA is purified both before and after capping and tailing.

In some embodiments, the mRNA is purified either before or after or both before and after capping and tailing, by centrifugation.

In some embodiments, the mRNA is purified either before or after or both before and after capping and tailing, by filtration.

In some embodiments, the mRNA is purified either before or after or both before and after capping and tailing, by Tangential Flow Filtration (TFF).

In some embodiments, the mRNA is purified either before or after or both before and after capping and tailing by chromatography.

Characterization of mRNA

Full-length or abortive transcripts of mRNA may be detected and quantified using any methods available in the art. In some embodiments, the synthesized mRNA molecules are detected using blotting, capillary electrophoresis, chromatography, fluorescence, gel electrophoresis, HPLC, silver stain, spectroscopy, ultraviolet (UV), or UPLC, or a combination thereof. Other detection methods known in the art are included in the present invention. In some embodiments, the synthesized mRNA molecules are detected using UV absorption spectroscopy with separation by capillary electrophoresis. In some embodiments, mRNA is first denatured by a Glyoxal dye before gel electrophoresis (“Glyoxal gel electrophoresis”). In some embodiments, synthesized mRNA is characterized before capping or tailing. In some embodiments, synthesized mRNA is characterized after capping and tailing.

In some embodiments, mRNA generated by the method disclosed herein comprises less than 10%, less than 9%, less than 8%, less than 7%, less than 6%, less than 5%, less than 4%, less than 3%, less than 2%, less than 1%, less than 0.5%, less than 0.1% impurities other than full length mRNA. The impurities include IVT contaminants, e.g., proteins, enzymes, free nucleotides and/or shortmers.

In some embodiments, mRNA produced according to the invention is substantially free of shortmers or abortive transcripts. In particular, mRNA produced according to the invention contains undetectable level of shortmers or abortive transcripts by capillary electrophoresis or Glyoxal gel electrophoresis. As used herein, the term “shortmers” or “abortive transcripts” refers to any transcripts that are less than full-length. In some embodiments, “shortmers” or “abortive transcripts” are less than 100 nucleotides in length, less than 90, less than 80, less than 70, less than 60, less than 50, less than 40, less than 30, less than 20, or less than 10 nucleotides in length. In some embodiments, shortmers are detected or quantified after adding a 5′-cap, and/or a 3′-poly A tail.

mRNA Solution

In some embodiments, mRNA may be provided in a solution to be mixed with a lipid solution such that the mRNA may be encapsulated in lipid nanoparticles. A suitable mRNA solution may be any aqueous solution containing mRNA to be encapsulated at various concentrations. For example, a suitable mRNA solution may contain an mRNA at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, or 1.0 mg/ml. In some embodiments, a suitable mRNA solution may contain an mRNA at a concentration ranging from about 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml. In some embodiments, a suitable mRNA solution may contain an mRNA at a concentration up to about 5.0 mg/ml, 4.0 mg/ml, 3.0 mg/ml, 2.0 mg/ml, 1.0 mg/ml, 0.09 mg/ml, 0.08 mg/ml, 0.07 mg/ml, 0.06 mg/ml, or 0.05 mg/ml.

Typically, a suitable mRNA solution may also contain a buffering agent and/or salt. Generally, buffering agents can include HEPES, ammonium sulfate, sodium bicarbonate, sodium citrate, sodium acetate, potassium phosphate and sodium phosphate. In some embodiments, suitable concentration of the buffering agent may range from about 0.1 mM to 100 mM, 0.5 mM to 90 mM, 1.0 mM to 80 mM, 2 mM to 70 mM, 3 mM to 60 mM, 4 mM to 50 mM, 5 mM to 40 mM, 6 mM to 30 mM, 7 mM to 20 mM, 8 mM to 15 mM, or 9 to 12 mM. In some embodiments, suitable concentration of the buffering agent is or greater than about 0.1 mM, 0.5 mM, 1 mM, 2 mM, 4 mM, 6 mM, 8 mM, 10 mM, 15 mM, 20 mM, 25 mM, 30 mM, 35 mM, 40 mM, 45 mM, or 50 mM.

Exemplary salts can include sodium chloride, magnesium chloride, and potassium chloride. In some embodiments, suitable concentration of salts in an mRNA solution may range from about 1 mM to 500 mM, 5 mM to 400 mM, 10 mM to 350 mM, 15 mM to 300 mM, 20 mM to 250 mM, 30 mM to 200 mM, 40 mM to 190 mM, 50 mM to 180 mM, 50 mM to 170 mM, 50 mM to 160 mM, 50 mM to 150 mM, or 50 mM to 100 mM. Salt concentration in a suitable mRNA solution is or greater than about 1 mM, 5 mM, 10 mM, 20 mM, 30 mM, 40 mM, 50 mM, 60 mM, 70 mM, 80 mM, 90 mM, or 100 mM.

In some embodiments, a suitable mRNA solution may have a pH ranging from about 3.5-6.5, 3.5-6.0, 3.5-5.5, 3.5-5.0, 3.5-4.5, 4.0-5.5, 4.0-5.0, 4.0-4.9, 4.0-4.8, 4.0-4.7, 4.0-4.6, or 4.0-4.5. In some embodiments, a suitable mRNA solution may have a pH of or no greater than about 3.5, 4.0, 4.1, 4.2, 4.3, 4.4, 4.5, 4.6, 4.7, 4.8, 4.9, 5.0, 5.2, 5.4, 5.6, 5.8, 6.0, 6.1, 6.3, and 6.5.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps.

Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of or greater than about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

### Delivery Vehicles

The stable lipid nanoparticles formulations described here are suitable as delivery vehicles for mRNA.

As used herein, the terms “delivery vehicle,” “transfer vehicle,” “nanoparticle” or grammatical equivalent, are used interchangeably.

Delivery vehicles can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition. A particular delivery vehicle is selected based upon its ability to facilitate the transfection of a nucleic acid to a target cell.

Liposomal Delivery Vehicles

In some embodiments, a suitable delivery vehicle is a liposomal delivery vehicle, e.g., a lipid nanoparticle. As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). In the context of the present invention, a liposomal delivery vehicle typically serves to transport a desired mRNA to a target cell or tissue. In some embodiments, a nanoparticle delivery vehicle is a liposome. In some embodiments, a liposome comprises one or more cationic lipids, one or more non-cationic lipids, one or more cholesterol-based lipids and one or more PEG-modified lipids. In some embodiments, a liposome comprises no more than three distinct lipid components. In some embodiments, one distinct lipid component is a sterol-based cationic lipid.

Cationic Lipids

As used herein, the phrase “cationic lipids” refers to any of a number of lipid species that have a net positive charge at a selected pH, such as physiological pH.

Suitable cationic lipids for use in the compositions and methods of the invention include the cationic lipids as described in International Patent Publication WO 2010/144740, which is incorporated herein by reference. In certain embodiments, the compositions and methods of the present invention include a cationic lipid, (6Z,9Z,28Z,31Z)-heptatriaconta-6,9,28,31-tetraen-19-yl 4-(dimethylamino) butanoate, having a compound structure of:

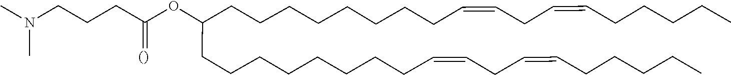

and pharmaceutically acceptable salts thereof.

Other suitable cationic lipids for use in the compositions and methods of the present invention include ionizable cationic lipids as described in International Patent Publication WO 2013/149140, which is incorporated herein by reference. In some embodiments, the compositions and methods of the present invention include a cationic lipid of one of the following formulas:

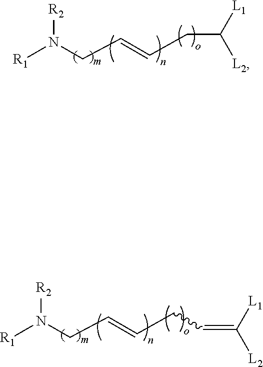

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In certain embodiments, the compositions and methods of the present invention include the cationic lipid (15Z, 18Z)-N,N-dimethyl-6-(9Z,12Z)-octadeca-9,12-dien-1-yl) tetracosa-15,18-dien-1-amine (“HGT5000”), having a compound structure of:

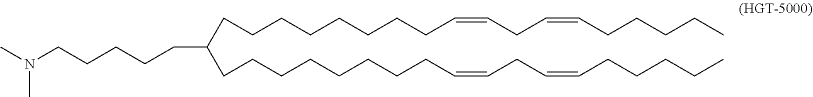 [SMILES: CCCCC/C=C\C/C=C\CCCCCCCCC(CCCCCCCC/C=C\C/C=C\CCCCC)CCCCCN(C)C]

and pharmaceutically acceptable salts thereof. In certain embodiments, the compositions and methods of the present invention include the cationic lipid (15Z, 18Z)-N,N-dimethyl-6-((9Z,12Z)-octadeca-9,12-dien-1-yl) tetracosa-4,15,18-trien-1-amine (“HGT5001”), having a compound structure of:

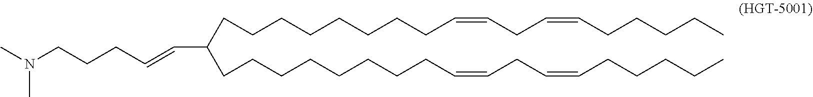 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In certain embodiments, the compositions and methods of the present invention include the cationic lipid and (15Z,18Z)-N,N-dimethyl-6-((9Z,12Z)-octadeca-9,12-dien-1-yl) tetracosa-5,15,18-trien-1-amine (“HGT5002”), having a compound structure of:

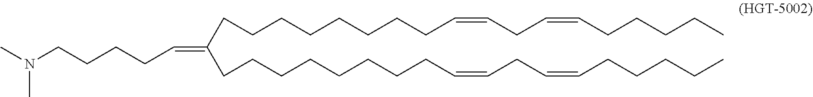 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the compositions and methods of the invention include cationic lipids described as aminoalcohol lipidoids in International Patent Publication WO 2010/053572, which is incorporated herein by reference. In certain embodiments, the compositions and methods of the present invention include a cationic lipid having a compound structure of:

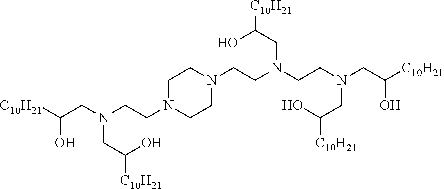

and pharmaceutically acceptable salts thereof.

Other suitable cationic lipids for use in the compositions and methods of the invention include the cationic lipids as described in International Patent Publication WO 2016/118725, which is incorporated herein by reference. In certain embodiments, the compositions and methods of the present invention include a cationic lipid having a compound structure of:

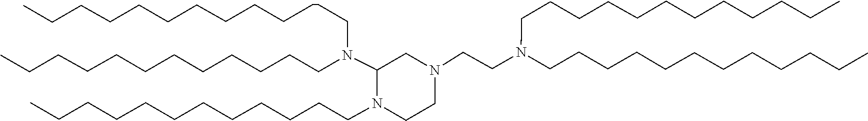 [SMILES: CCCCCCCCCCCCCN(CCCCCCCCCCCC)CCN1CCN(CCCCCCCCCCCC)C(N(CCCCCCCCCCCC)CCCCCCCCCCCCC)C1]

and pharmaceutically acceptable salts thereof.

Other suitable cationic lipids for use in the compositions and methods of the invention include the cationic lipids as described in International Patent Publication WO 2016/118724, which is incorporated herein by reference. In certain embodiments, the compositions and methods of the present invention include a cationic lipid having a compound structure of:

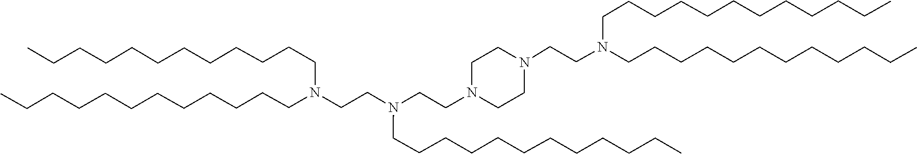 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the compositions and methods of the invention include the cationic lipids as described in International Patent Publications WO 2013/063468 and WO 2016/205691, each of which are incorporated herein by reference. In some embodiments, the compositions and methods of the present invention include a cationic lipid of the following formula:

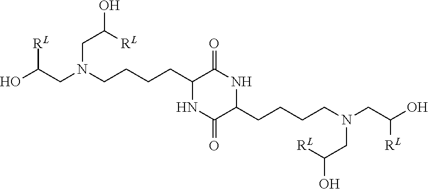

or pharmaceutically acceptable salts thereof, wherein each instance of R^{L} is independently optionally substituted C₆-C₄₀ alkenyl. In certain embodiments, the compositions and methods of the present invention include a cationic lipid having a compound structure of:

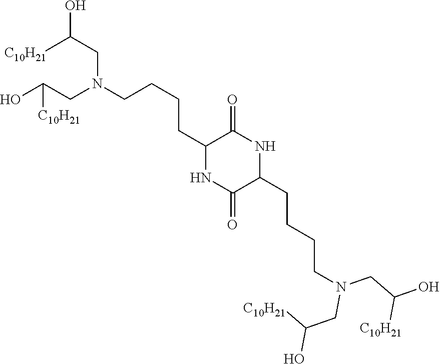

and pharmaceutically acceptable salts thereof. In certain embodiments, the compositions and methods of the present invention include a cationic lipid having a compound structure of:

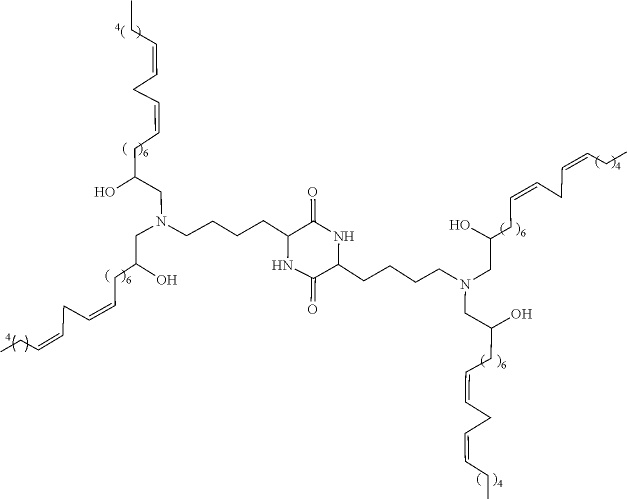

and pharmaceutically acceptable salts thereof. In certain embodiments, the compositions and methods of the present invention include a cationic lipid having a compound structure of:

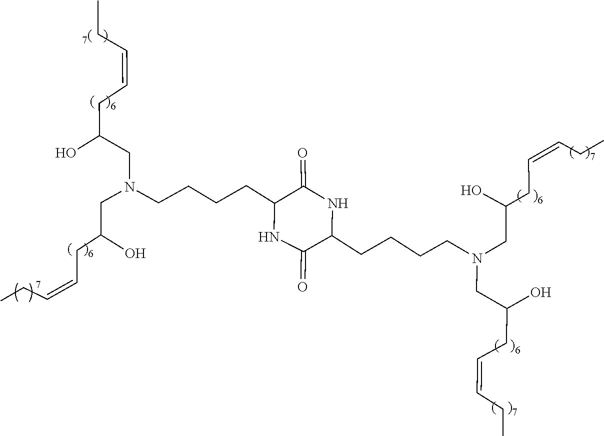

and pharmaceutically acceptable salts thereof. In certain embodiments, the compositions and methods of the present invention include a cationic lipid having a compound structure of:

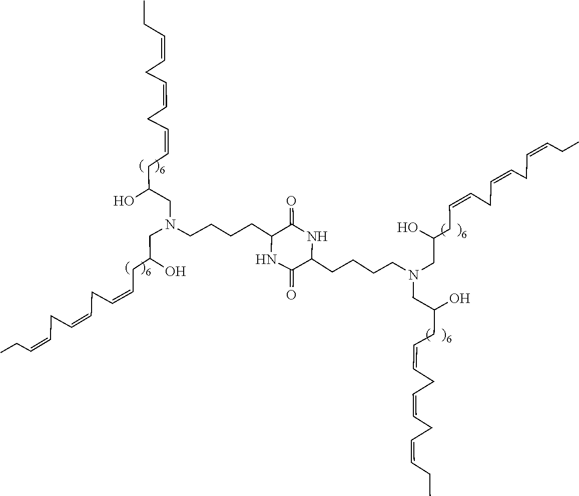

and pharmaceutically acceptable salts thereof.

Other suitable cationic lipids for use in the compositions and methods of the invention include the cationic lipids as described in International Patent Publication WO 2015/184256, which is incorporated herein by reference. In some embodiments, the compositions and methods of the present invention include a cationic lipid of the following formula:

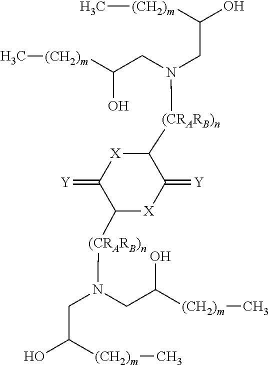

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each RB is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In certain embodiments, the compositions and methods of the present invention include a cationic lipid, “Target 23”, having a compound structure of:

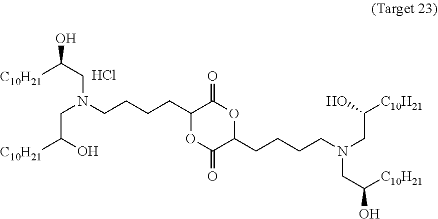

and pharmaceutically acceptable salts thereof.

Other suitable cationic lipids for use in the compositions and methods of the invention include the cationic lipids as described in International Patent Publication WO 2016/004202, which is incorporated herein by reference. In some embodiments, the compositions and methods of the present invention include a cationic lipid having the compound structure:

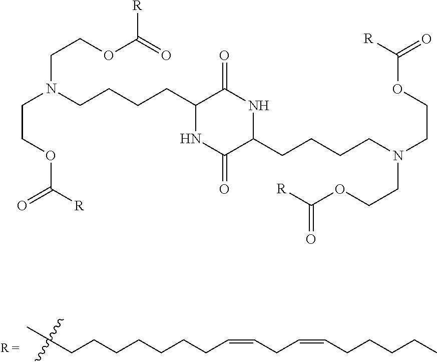

or a pharmaceutically acceptable salt thereof. In some embodiments, the compositions and methods of the present invention include a cationic lipid having the compound structure:

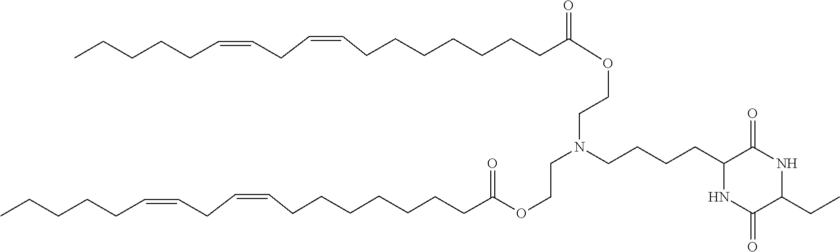 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the compositions and methods of the present invention include a cationic lipid having the compound structure:

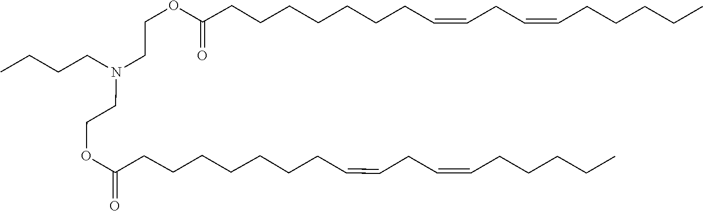 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the compositions and methods of the present invention include cationic lipids as described in U.S. Provisional Patent Application No. 62/758,179, which is incorporated herein by reference. In some embodiments, the compositions and methods of the present invention include a cationic lipid of the following formula:

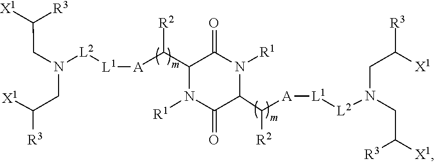

or a pharmaceutically acceptable salt thereof, wherein each R¹ and R² is independently H or C₁-C₆ aliphatic; each m is independently an integer having a value of 1 to 4; each A is independently a covalent bond or arylene; each L¹ is independently an ester, thioester, disulfide, or anhydride group; each L² is independently C₂-C₁₀ aliphatic; each X¹ is independently H or OH; and each R³ is independently C₆-C₂₀ aliphatic. In some embodiments, the compositions and methods of the present invention include a cationic lipid of the following formula:

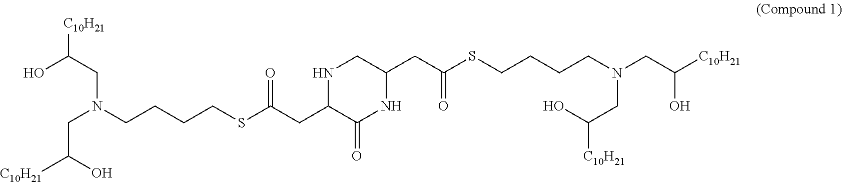

or a pharmaceutically acceptable salt thereof. In some embodiments, the compositions and methods of the present invention include a cationic lipid of the following formula:

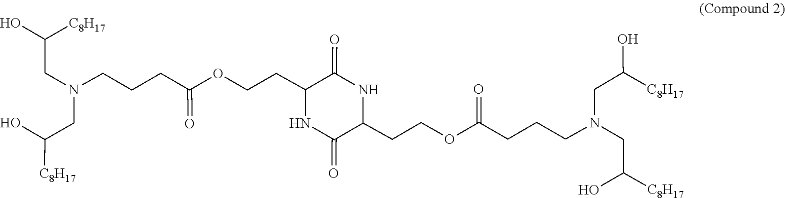

or a pharmaceutically acceptable salt thereof. In some embodiments, the compositions and methods of the present invention include a cationic lipid of the following formula:

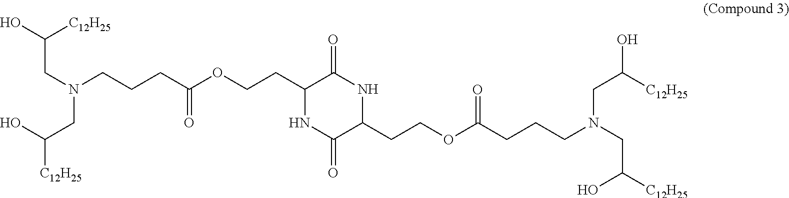

or a pharmaceutically acceptable salt thereof.

Other suitable cationic lipids for use in the compositions and methods of the present invention include the cationic lipids as described in J. McClellan, M. C. King, Cell 2010, 141, 210-217 and in Whitehead et al., Nature Communications (2014) 5:4277, which is incorporated herein by reference. In certain embodiments, the cationic lipids of the compositions and methods of the present invention include a cationic lipid having a compound structure of:

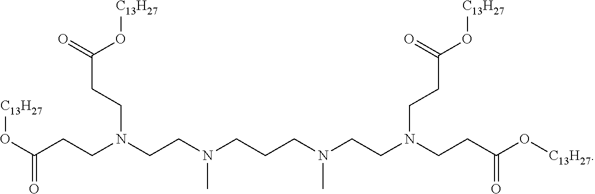

and pharmaceutically acceptable salts thereof.

Other suitable cationic lipids for use in the compositions and methods of the invention include the cationic lipids as described in International Patent Publication WO 2015/199952, which is incorporated herein by reference. In some embodiments, the compositions and methods of the present invention include a cationic lipid having the compound structure:

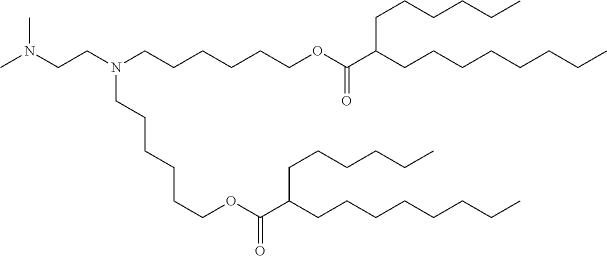 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

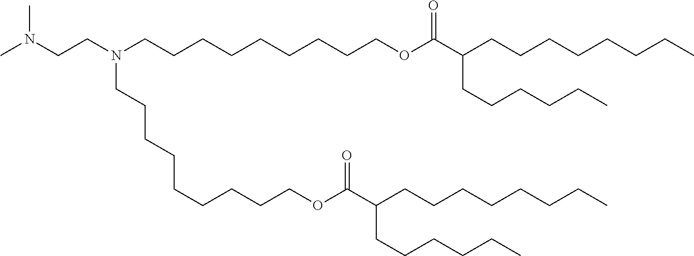 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

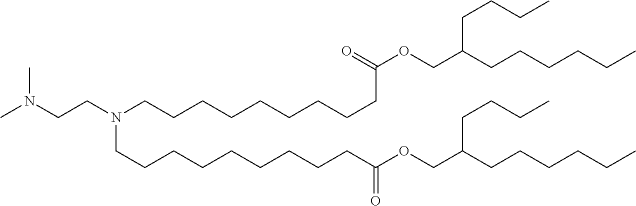 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the compositions and methods of the present invention include a cationic lipid having the compound structure:

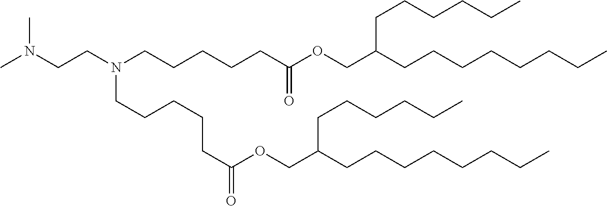 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

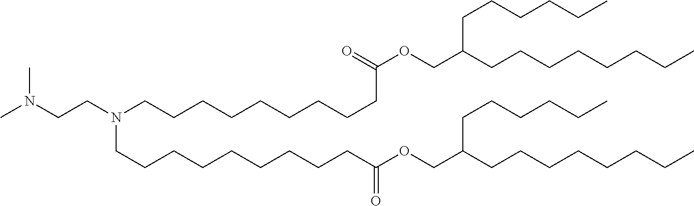 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

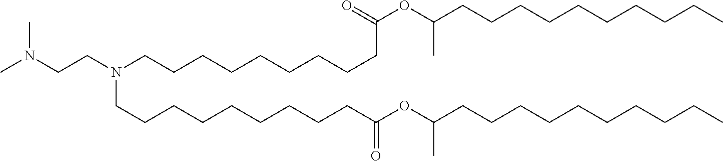 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the compositions and methods of the present invention include a cationic lipid having the compound structure:

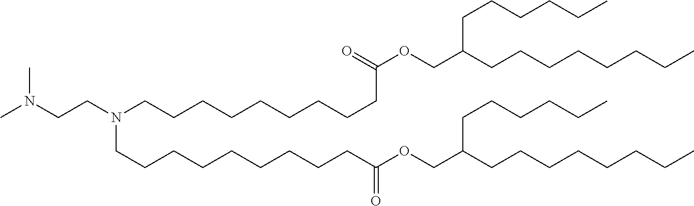 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

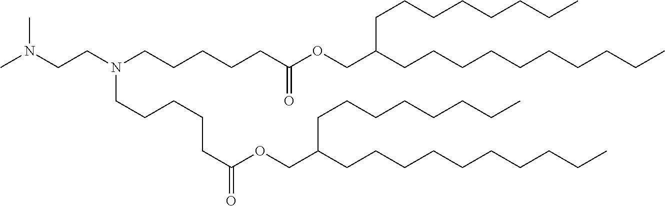 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

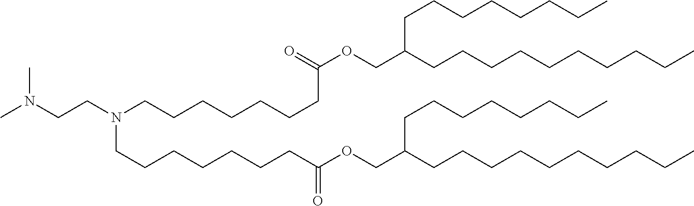 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

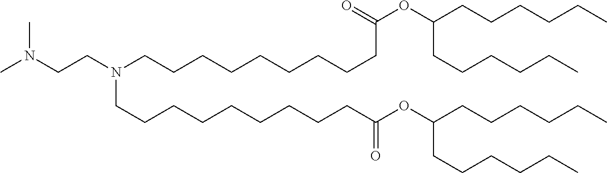 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the compositions and methods of the present invention include a cationic lipid having the compound structure:

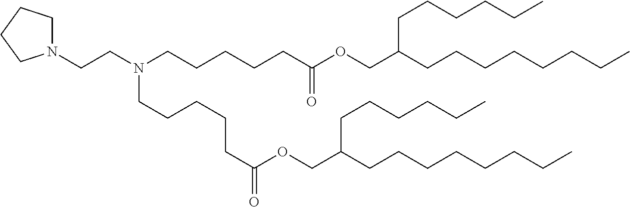 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the compositions and methods of the present invention include a cationic lipid having the compound structure:

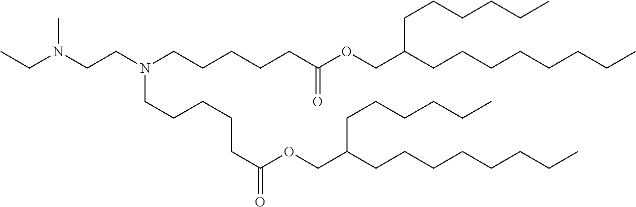 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the compositions and methods of the present invention include a cationic lipid having the compound structure:

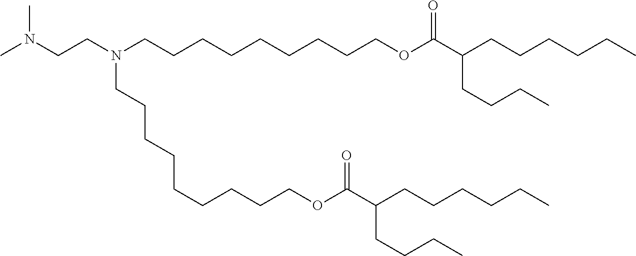 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the compositions and methods of the invention include the cationic lipids as described in International Patent Publication WO 2017/004143, which is incorporated herein by reference. In some embodiments, the compositions and methods of the present invention include a cationic lipid having the compound structure:

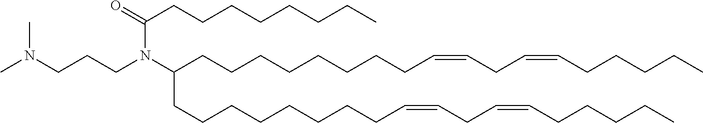 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the compositions and methods of the present invention include a cationic lipid having the compound structure:

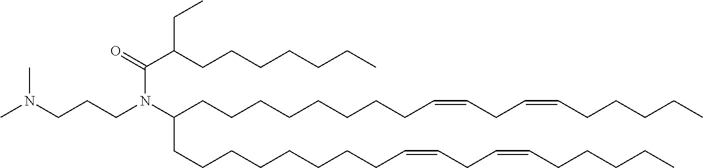 [SMILES: CCCCC/C=C\C/C=C\CCCCCCCCC(CCCCCCCC/C=C\C/C=C\CCCCC)N(CCCN(C)C)C(=O)C(CC)CCCCCCC]

and pharmaceutically acceptable salts thereof. In some embodiments, the compositions and methods of the present invention include a cationic lipid having the compound structure:

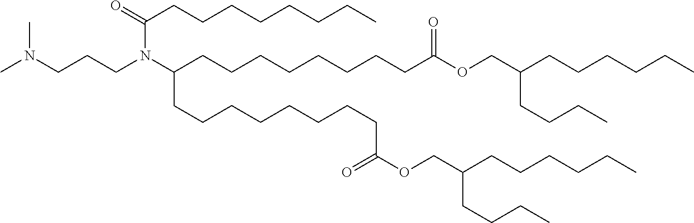 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

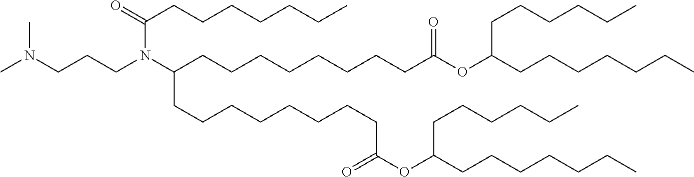 [SMILES: CCCCCCCC(=O)N(CCCN(C)C)C(CCCCCCCCC(=O)OC(CCCCCC)CCCCCCC)CCCCCCCCC(=O)OC(CCCCCC)CCCCCCC]

and pharmaceutically acceptable salts thereof. In some embodiments, the compositions and methods of the present invention include a cationic lipid having the compound structure:

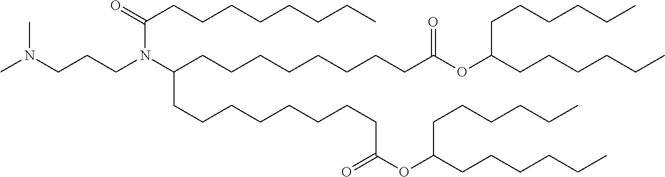 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

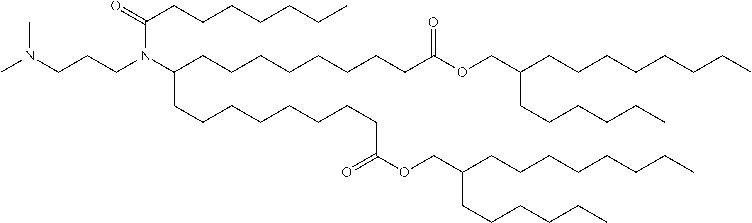 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

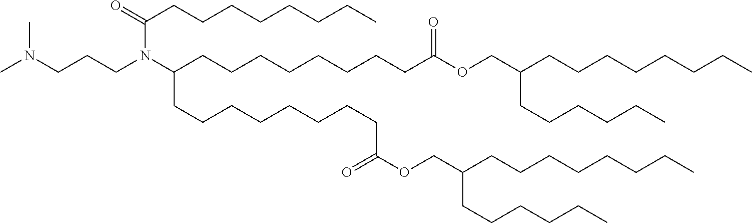 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the compositions and methods of the present invention include a cationic lipid having the compound structure:

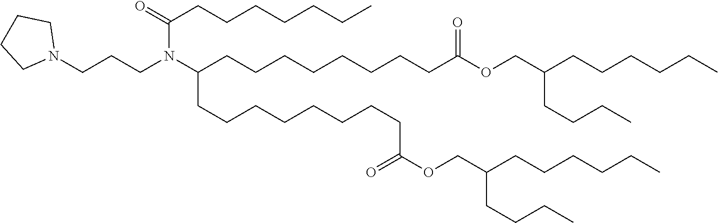 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

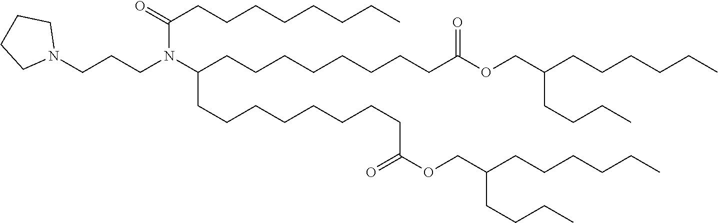 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

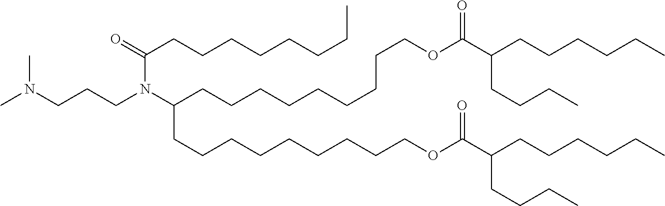 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the compositions and methods of the present invention include a cationic lipid having the compound structure:

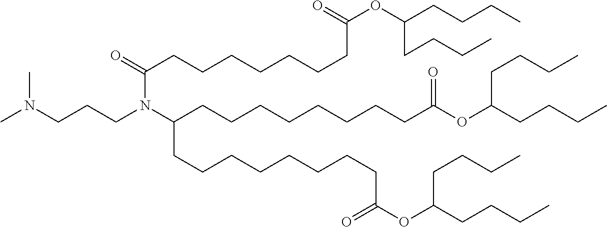 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the compositions and methods of the present invention include a cationic lipid having the compound structure:

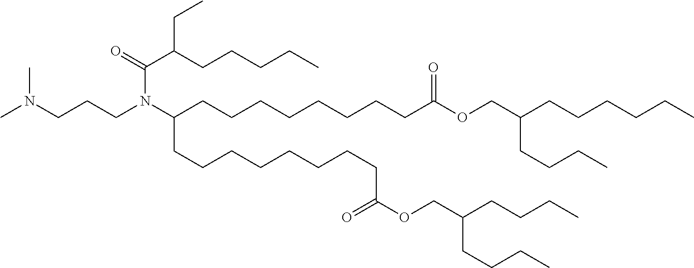 [SMILES: CCCCCCC(CCCC)COC(=O)CCCCCCCCC(CCCCCCCCC(=O)OCC(CCCC)CCCC)N(CCCN(C)C)C(=O)C(CC)CCCCC]

and pharmaceutically acceptable salts thereof. In some embodiments, the compositions and methods of the present invention include a cationic lipid having the compound structure:

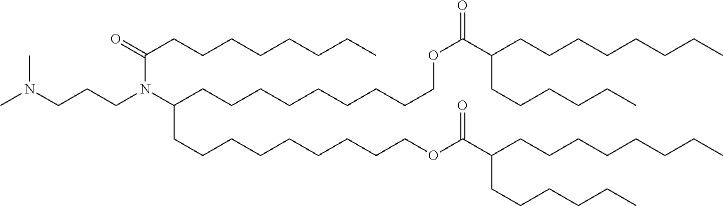 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the compositions and methods of the present invention include a cationic lipid having the compound structure:

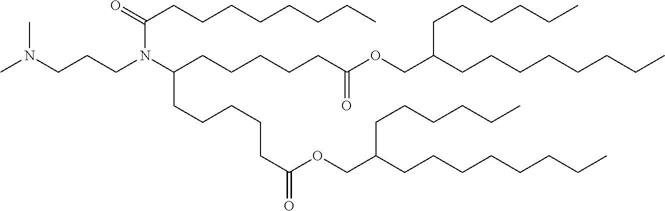 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the compositions and methods of the present invention include a cationic lipid having the compound structure:

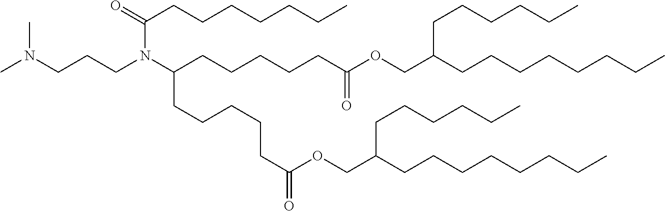 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

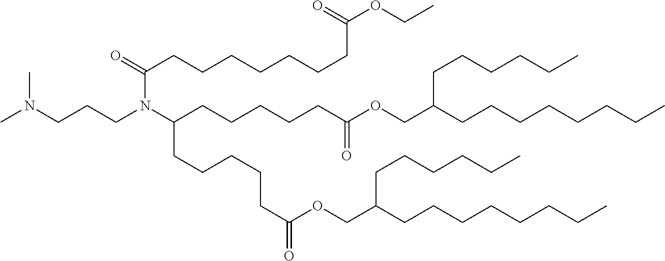 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the compositions and methods of the present invention include a cationic lipid having the compound structure:

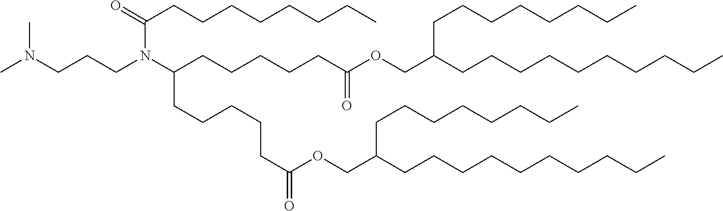 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the compositions and methods of the invention include the cationic lipids as described in International Patent Publication WO 2017/075531, which is incorporated herein by reference. In some embodiments, the compositions and methods of the present invention include a cationic lipid of the following formula:

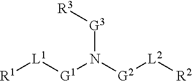

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, —NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable cationic lipids for use in the compositions and methods of the invention include the cationic lipids as described in International Patent Publication WO 2017/117528, which is incorporated herein by reference. In some embodiments, the compositions and methods of the present invention include a cationic lipid having the compound structure:

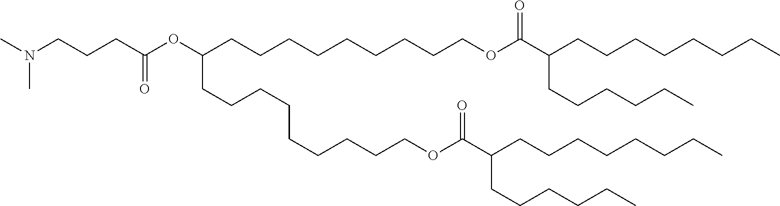 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the compositions and methods of the present invention include a cationic lipid having the compound structure:

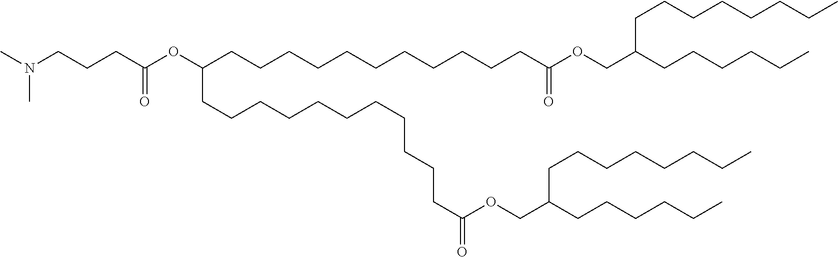 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the compositions and methods of the present invention include a cationic lipid having the compound structure:

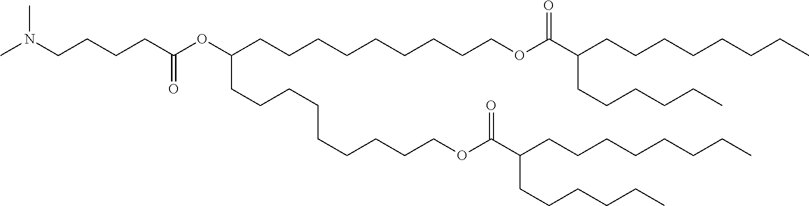 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the compositions and methods of the invention include the cationic lipids as described in International Patent Publication WO 2017/049245, which is incorporated herein by reference. In some embodiments, the cationic lipids of the compositions and methods of the present invention include a compound of one of the following formulas:

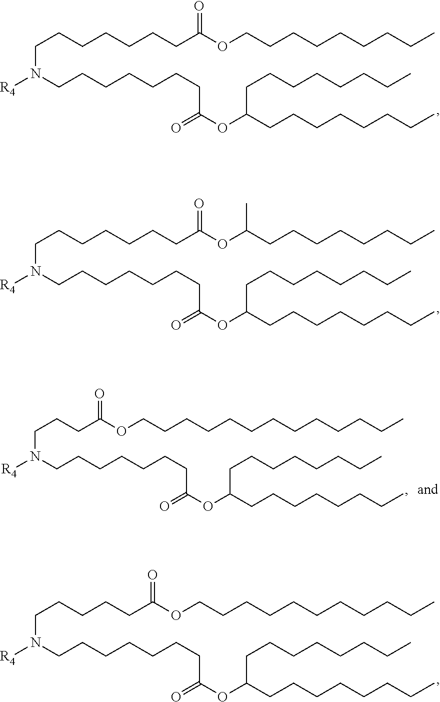

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In certain embodiments, the compositions and methods of the present invention include a cationic lipid having a compound structure of:

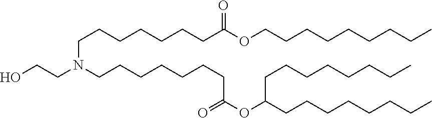 [SMILES: CCCCCCCCCOC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

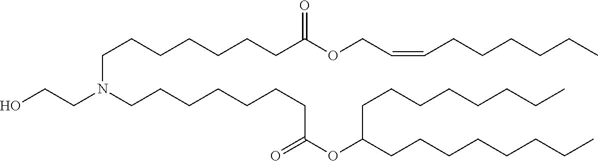 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

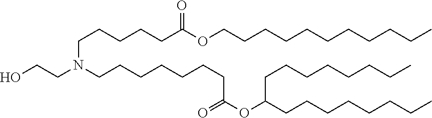 [SMILES: CCCCCCCCCCCOC(=O)CCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

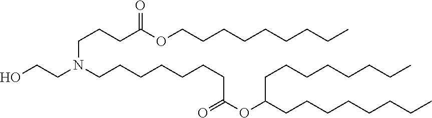 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the compositions and methods of the invention include the cationic lipids as described in International Patent Publications WO 2017/173054 and WO 2015/095340, each of which is incorporated herein by reference. In certain embodiments, the compositions and methods of the present invention include a cationic lipid having a compound structure of:

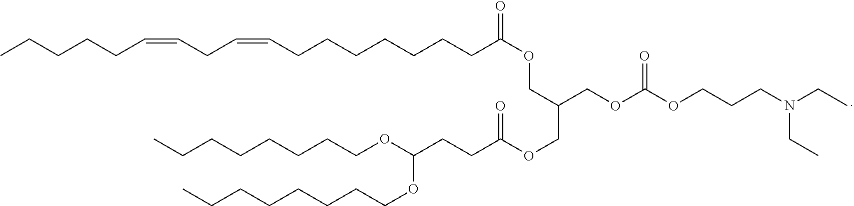 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof. In certain embodiments, the compositions and methods of the present invention include a cationic lipid having a compound structure of:

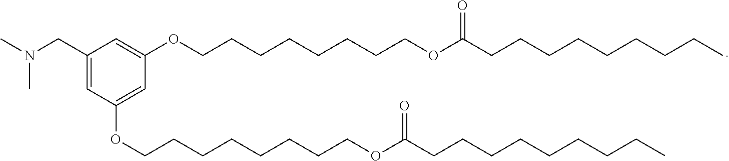 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In certain embodiments, the compositions and methods of the present invention include a cationic lipid having a compound structure of:

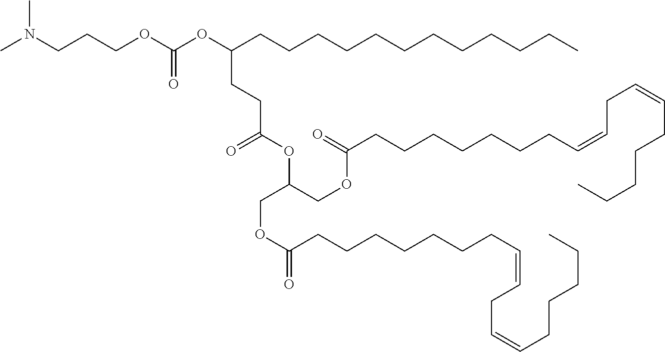 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In certain embodiments, the compositions and methods of the present invention include a cationic lipid having a compound structure of:

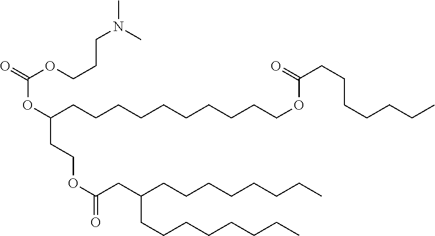 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the compositions and methods of the present invention include cleavable cationic lipids as described in International Patent Publication WO 2012/170889, which is incorporated herein by reference. In some embodiments, the compositions and methods of the present invention include a cationic lipid of the following formula:

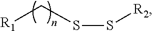

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

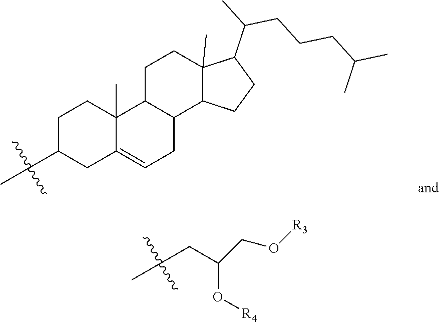

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the compositions and methods of the present invention include a cationic lipid, “HGT4001”, having a compound structure of:

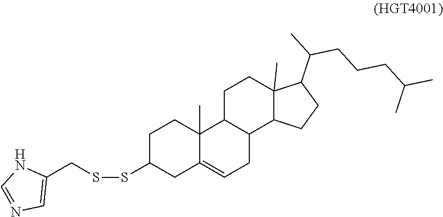 [SMILES: CC(C)CCCC(C)C1CCC2C3CC=C4CC(SSCc5cnc[nH]5)CCC4(C)C3CCC12C]

and pharmaceutically acceptable salts thereof. In certain embodiments, the compositions and methods of the present invention include a cationic lipid, “HGT4002” (also referred to herein as “Guan-SS-Chol”), having a compound structure of:

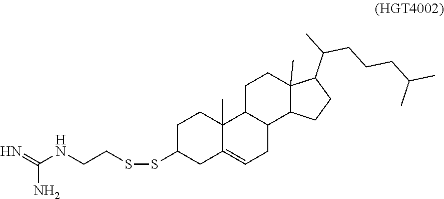 [SMILES: CC(C)CCCC(C)C1CCC2C3CC=C4CC(SSCCNC(=N)N)CCC4(C)C3CCC12C]

and pharmaceutically acceptable salts thereof. In certain embodiments, the compositions and methods of the present invention include a cationic lipid, “HGT4003”, having a compound structure of:

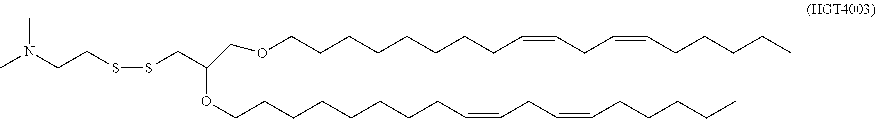 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In certain embodiments, the compositions and methods of the present invention include a cationic lipid, “HGT4004”, having a compound structure of:

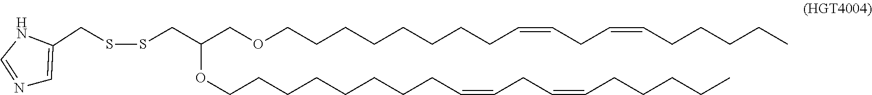 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In certain embodiments, the compositions and methods of the present invention include a cationic lipid “HGT4005”, having a compound structure of:

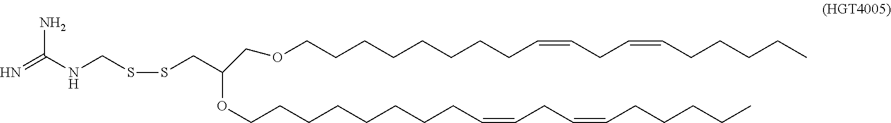 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the compositions and methods of the present invention include cleavable cationic lipids as described in U.S. Provisional Patent Application No. 62/672,194, filed May 16, 2018, and incorporated herein by reference. In certain embodiments, the compositions and methods of the present invention include a cationic lipid that is any of general formulas or any of structures (1a)-(21a) and (1b)-(21b) and (22)-(237) described in U.S. Provisional Patent Application No. 62/672,194. In certain embodiments, the compositions and methods of the present invention include a cationic lipid that has a structure according to Formula (I′),

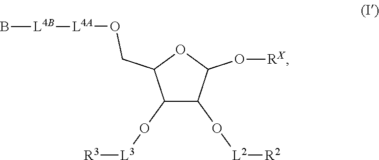

wherein:
R^{X} is independently —H, or —L¹—R¹, or —L^{5A}—L^{5B}—B′;
each of L¹, L², and L³ is independently a covalent bond, —C(O)—, —C(O)O—, —C(O)S—, or —C(O)NR^{L}—;
each L^{4A} and L^{5A} is independently —C(O)—, —C(O)O—, or —C(O)NR^{L}—;
each L^{4B} and L^{5B} is independently C₁-C₂₀ alkylene; C₂-C₂₀ alkenylene; or C₂-C₂₀ alkynylene;
each B and B′ is NR⁴R⁵ or a 5- to 10-membered nitrogen-containing heteroaryl;
each R¹, R², and R³ is independently C₆-C₃₀ alkyl, C₆-C₃₀ alkenyl, or C₆-C₃₀ alkynyl;
each R⁴ and R⁵ is independently hydrogen, C₁-C₁₀ alkyl; C₂-C₁₀ alkenyl; or C₂-C₁₀ alkynyl; and
each R^{L} is independently hydrogen, C₁-C₂₀ alkyl, C₂-C₂₀ alkenyl, or C₂-C₂₀ alkynyl.
   In certain embodiments, the compositions and methods of the present invention include a cationic lipid that is Compound (139) of 62/672,194, having a compound structure of:

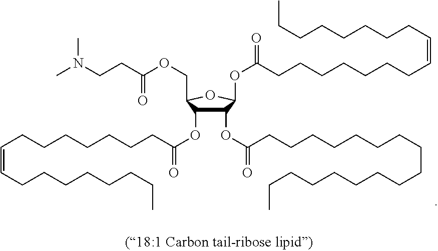

In some embodiments, the compositions and methods of the present invention include the cationic lipid, N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the compositions and methods of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.'l Acad. Sci. 86, 6982 (1989), U.S. Pat. No. 5,171,678; U.S. Pat. No. 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1 ,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethy 1-1-(cis,cis-9′,1-2′-octadecadienoxy)propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N, N-dimethyl-3-[(9Z, 12Z)-octadeca-9, 12-dien-1-yloxy]propane-1-amine (“Octyl-CLinDMA”); (2R)-2-((8-[(3beta)-cholest-5-en-3-yloxy]octyl)oxy)-N, N-dimethyl-3-[(9Z, 12Z)-octadeca-9,12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-[(9Z, 12Z)-octadeca-9, 12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,12-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al., Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety. In some embodiments, one or more cationic lipids suitable for the compositions and methods of the present invention include 2,2-Dilinoley1-4-dimethylaminoethy 1-[1,3]-dioxolane (“XTC”); (3aR,5s,6aS)-N,N-dimethyl-2,2-di((9Z,12Z)-octadeca-9,12-dienyl)tetrahydro-3aH-cyclopenta[d] [1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, one or more cationic lipids suitable for the compositions and methods of the present invention include a cationic lipid that is TL1-04D-DMA, having a compound structure of:

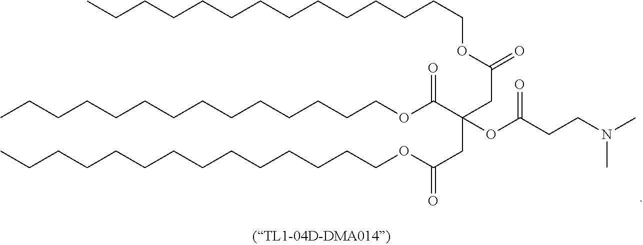 [SMILES: CCCCCCCCCCCCCCOC(=O)CC(CC(=O)OCCCCCCCCCCCCCC)(OC(=O)CCN(C)C)C(=O)OCCCCCCCCCCCCCC]

In some embodiments, one or more cationic lipids suitable for the compositions and methods of the present invention include a cationic lipid that is GL-TES-SA-DME-E18-2, having a compound structure of:

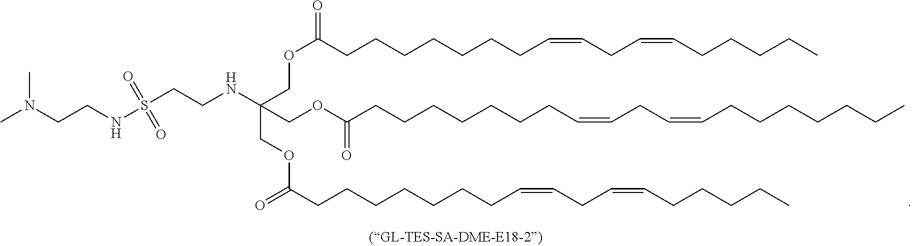 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCCCC)NCCS(=O)(=O)NCCN(C)C]

In some embodiments, one or more cationic lipids suitable for the compositions and methods of the present invention include a cationic lipid that is SY-3-E14-DMAPr, having a compound structure of:

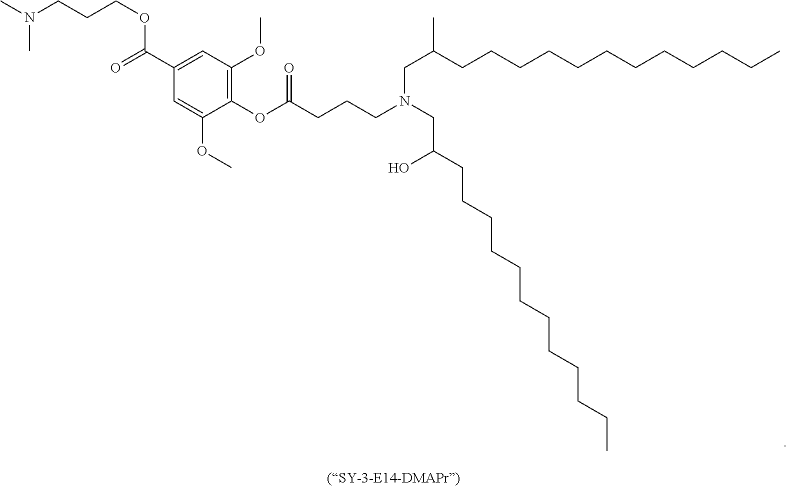 [SMILES: CCCCCCCCCCCCC(C)CN(CCCC(=O)Oc1c(OC)cc(C(=O)OCCCN(C)C)cc1OC)CC(O)CCCCCCCCCCCC]

In some embodiments, one or more cationic lipids suitable for the compositions and methods of the present invention include a cationic lipid that is TL1-01D-DMA, having a compound structure of:

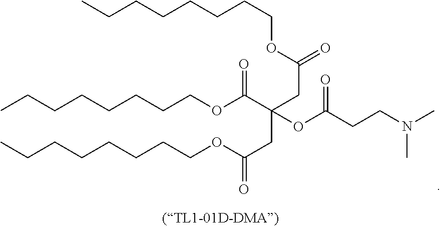 [SMILES: CCCCCCCCOC(=O)CC(CC(=O)OCCCCCCCC)(OC(=O)CCN(C)C)C(=O)OCCCCCCCC]

In some embodiments, one or more cationic lipids suitable for the compositions and methods of the present invention include a cationic lipid that is TL1-10D-DMA, having a compound structure of:

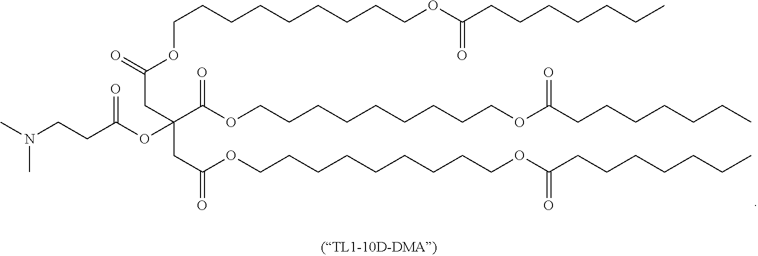 [SMILES: CCCCCCCC(=O)OCCCCCCCCCOC(=O)CC(CC(=O)OCCCCCCCCCOC(=O)CCCCCCC)(OC(=O)CCN(C)C)C(=O)OCCCCCCCCCOC(=O)CCCCCCC]

In some embodiments, one or more cationic lipids suitable for the compositions and methods of the present invention include a cationic lipid that is GL-TES-SA-DMP-E18-2, having a compound structure of:

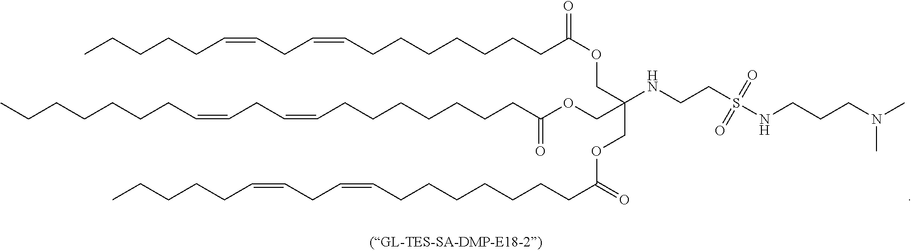 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCCCC)NCCS(=O)(=O)NCCCN(C)C]

In some embodiments, one or more cationic lipids suitable for the compositions and methods of the present invention include a cationic lipid that is HEP-E4-E10, having a compound structure of:

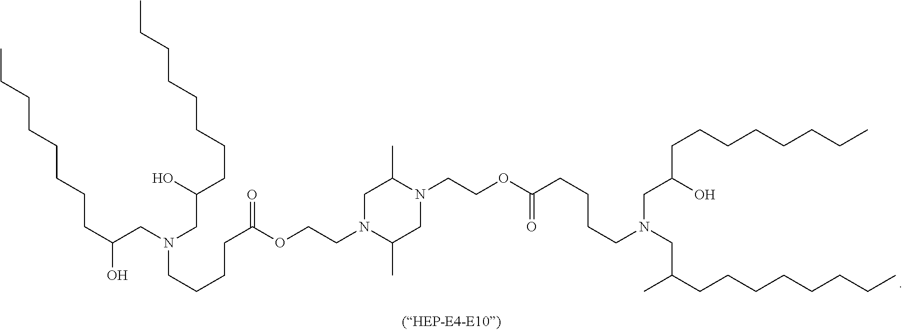 [SMILES: CCCCCCCCC(C)CN(CCCCC(=O)OCCN1CC(C)N(CCOC(=O)CCCCN(CC(O)CCCCCCCC)CC(O)CCCCCCCC)CC1C)CC(O)CCCCCCCC]

In some embodiments, one or more cationic lipids suitable for the compositions and methods of the present invention include a cationic lipid that is HEP-E3-E10, having a compound structure of:

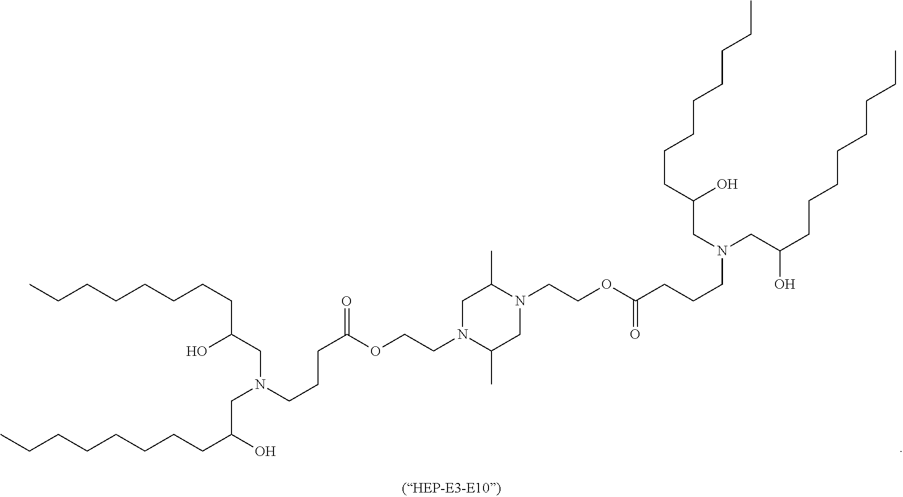 [SMILES: CCCCCCCCC(O)CN(CCCC(=O)OCCN1CC(C)N(CCOC(=O)CCCN(CC(O)CCCCCCCC)CC(O)CCCCCCCC)CC1C)CC(O)CCCCCCCC]

In some embodiments, the compositions of the present invention include one or more cationic lipids that constitute at least about 5%, 10%, 20%, 30%, 35%, 40%, 45%, 50%, 55%, 60%, 65%, or 70%, measured by weight, of the total lipid content in the composition, e.g., a lipid nanoparticle. In some embodiments, the compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the composition, e.g., a lipid nanoparticle.

Non-Cationic/Helper Lipids

In some embodiments, provided liposomes contain one or more non-cationic (“helper”) lipids. As used herein, the phrase “non-cationic lipid” refers to any neutral, zwitterionic or anionic lipid. As used herein, the phrase “anionic lipid” refers to any of a number of lipid species that carry a net negative charge at a selected H, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), di stearoyl-phosphatidyl-ethanolamine (DSPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof.

In some embodiments, such non-cationic lipids may be used alone, but are preferably used in combination with other lipids, for example, cationic lipids. In some embodiments, the non-cationic lipid may comprise a molar ratio of about 5% to about 90%, or about 10% to about 70% of the total lipid present in a liposome. In some embodiments, a non-cationic lipid is a neutral lipid, i.e., a lipid that does not carry a net charge in the conditions under which the composition is formulated and/or administered. In some embodiments, the percentage of non-cationic lipid in a liposome may be greater than 5%, greater than 10%, greater than 20%, greater than 30%, or greater than 40%.

Cholesterol-Based Lipids

In some embodiments, provided liposomes comprise one or more cholesterol-based lipids. For example, suitable cholesterol-based cationic lipids include, for example, DC-Choi (N,N-dimethyl-N-ethylcarboxamidocholesterol),1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE. In some embodiments, the cholesterol-based lipid may comprise a molar ration of about 2% to about 30%, or about 5% to about 20% of the total lipid present in a liposome. In some embodiments, the percentage of cholesterol-based lipid in the lipid nanoparticle may be greater than 5%, greater than 10%, greater than 20%, greater than 30%, or greater than 40%.

PEG-Modified Lipids

The use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipid formulations together which comprise the transfer vehicle (e.g., a lipid nanoparticle). Contemplated PEG-modified lipids include, but are not limited to, a polyethylene glycol chain of up to 5 kDa in length covalently attached to a lipid with alkyl chain(s) of C₆-C₂₀ length. The addition of such components may prevent complex aggregation and may also provide a means for increasing circulation lifetime and increasing the delivery of the lipid-nucleic acid composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in the liposomal transfer vehicle.

According to various embodiments, the selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the MCNA to be delivered. Additional considerations include, for example, the saturation of the alkyl chain, as well as the size, charge, pH, pKa, fusogenicity and toxicity of the selected lipid(s). Thus the molar ratios may be adjusted accordingly.

Polymers

In some embodiments, a suitable delivery vehicle is formulated using a polymer as a carrier, alone or in combination with other carriers including various lipids described herein. Thus, in some embodiments, liposomal delivery vehicles, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

Liposomes Suitable for Use with the Present Invention

A suitable liposome for the present invention may include one or more of any of the cationic lipids, non-cationic lipids, cholesterol lipids, PEG-modified lipids and/or polymers described herein at various ratios. As non-limiting examples, a suitable liposome formulation may include a combination selected from cKK-E12, DOPE, cholesterol and DMG-PEG2K; C12-200, DOPE, cholesterol and DMG-PEG2K; HGT4003, DOPE, cholesterol and DMG-PEG2K; ICE, DOPE, cholesterol and DMG-PEG2K; or ICE, DOPE, and DMG-PEG2K.

In various embodiments, cationic lipids (e.g., cKK-E12, C12-200, ICE, and/or HGT4003) constitute about 30-60% (e.g., about 30-55%, about 30-50%, about 30-45%, about 30-40%, about 35-50%, about 35-45%, or about 35-40%) of the liposome by molar ratio. In some embodiments, the percentage of cationic lipids (e.g., cKK-E12, C12-200, ICE, and/or HGT4003) is or greater than about 30%, about 35%, about 40%, about 45%, about 50%, about 55%, or about 60% of the liposome by molar ratio.

In some embodiments, the ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5.

In particular embodiments, a liposome for use with this invention comprises a lipid component consisting of a cationic lipid, a non-cationic lipid (e.g., DOPE or DEPE), a PEG-modified lipid (e.g., DMG-PEG2K), and optionally cholesterol. Cationic lipids particularly suitable for inclusion in such a liposome include GL-TES-SA-DME-E18-2, TL1-01D-DMA, SY-3-E14-DMAPr, TL1-10D-DMA, HGT4002 (also referred to herein as Guan-SS-Chol), GL-TES-SA-DMP-E18-2, HEP-E4-E10, HEP-E3-E10, and TL1-04D-DMA. These cationic lipids have been found to be particularly suitable for use in liposomes that are administered through pulmonary delivery via nebulization. Amongst these, HEP-E4-E10, HEP-E3-E10, GL-TES-SA-DME-E18-2, GL-TES-SA-DMP-E18-2, TL1-01D-DMA and TL1-04D-DMA performed particularly well.

Exemplary liposomes include one of GL-TES-SA-DME-E18-2, TL1-01D-DMA, SY-3-E14-DMAPr, TL1-10D-DMA, GL-TES-SA-DMP-E18-2, HEP-E4-E10, HEP-E3-E10 and TL1-04D-DMA as a cationic lipid component, DOPE as a non-cationic lipid component, cholesterol as a helper lipid component, and DMG-PEG2K as a PEG-modified lipid component. In some embodiments, the molar ratio of the cationic lipid to non-cationic lipid to cholesterol to PEG-modified lipid may be between about 30-60:25-35:20-30:1-15, respectively. In some embodiments, the molar ratio of cationic lipid to non-cationic lipid to cholesterol to PEG-modified lipid is approximately 40:30:20:10, respectively. In some embodiments, the molar ratio of cationic lipid to non-cationic lipid to cholesterol to PEG-modified lipid is approximately 40:30:25:5, respectively. In some embodiments, the molar ratio of cationic lipid to non-cationic lipid to cholesterol to PEG-modified lipid is approximately 40:32:25:3, respectively. In some embodiments, the molar ratio of cationic lipid to non-cationic lipid to cholesterol to PEG-modified lipid is approximately 50:25:20:5.

In some embodiments, the lipid component of a liposome particularly suitable for pulmonary delivery consists of HGT4002 (also referred to herein as Guan-SS-Chol), DOPE and DMG-PEG2K. In some embodiments, the molar ratio of cationic lipid to non-cationic lipid to PEG-modified lipid is approximately 60:35:5.

Ratio of Distinct Lipid Components

In embodiments where a lipid nanoparticle comprises three and no more than three distinct components of lipids, the ratio of total lipid content (i.e., the ratio of lipid component (1): lipid component (2): lipid component (3)) can be represented as x:y:z, wherein

(y+z)=100−x.

In some embodiments, each of “x,” “y,” and “z” represents molar percentages of the three distinct components of lipids, and the ratio is a molar ratio.

In some embodiments, each of “x,” “y,” and “z” represents weight percentages of the three distinct components of lipids, and the ratio is a weight ratio.

In some embodiments, lipid component (1), represented by variable “x,” is a sterol-based cationic lipid.

In some embodiments, lipid component (2), represented by variable “y,” is a helper lipid.

In some embodiments, lipid component (3), represented by variable “z” is a PEG lipid.

In some embodiments, variable “x,” representing the molar percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the molar percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the molar percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “x,” representing the weight percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the weight percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the weight percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “z,” representing the molar percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the molar percentage of lipid component (3) (e.g., a PEG lipid) is about 1%, 2%, 3%, 4%, 5%, 6%, 7%, 8%, 9%, 10%. In embodiments, variable “z,” representing the molar percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

In some embodiments, variable “z,” representing the weight percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the weight percentage of lipid component (3) (e.g., a PEG lipid) is about 1%, 2%, 3%, 4%, 5%, 6%, 7%, 8%, 9%, 10%. In embodiments, variable “z,” representing the weight percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

For compositions having three and only three distinct lipid components, variables “x,” “y,” and “z” may be in any combination so long as the total of the three variables sums to 100% of the total lipid content.

Formation of Liposomes Encapsulating mRNA

The liposomal transfer vehicles for use in the compositions of the invention can be prepared by various techniques which are presently known in the art. The liposomes for use in provided compositions can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Unilamellar vesicles (ULV) can then be formed by homogenization, sonication or extrusion of the multilamellar vesicles. In addition, unilamellar vesicles can be formed by detergent removal techniques.

In certain embodiments, provided compositions comprise a liposome wherein the mRNA is associated on both the surface of the liposome and encapsulated within the same liposome. For example, during preparation of the compositions of the present invention, cationic liposomes may associate with the mRNA through electrostatic interactions. For example, during preparation of the compositions of the present invention, cationic liposomes may associate with the mRNA through electrostatic interactions.

In some embodiments, the compositions and methods of the invention comprise mRNA encapsulated in a liposome. In some embodiments, the one or more mRNA species may be encapsulated in the same liposome. In some embodiments, the one or more mRNA species may be encapsulated in different liposomes. In some embodiments, the mRNA is encapsulated in one or more liposomes, which differ in their lipid composition, molar ratio of lipid components, size, charge (zeta potential), targeting ligands and/or combinations thereof. In some embodiments, the one or more liposome may have a different composition of sterol-based cationic lipids, neutral lipid, PEG-modified lipid and/or combinations thereof. In some embodiments the one or more liposomes may have a different molar ratio of cholesterol-based cationic lipid, neutral lipid, and PEG-modified lipid used to create the liposome.

The process of incorporation of a desired mRNA into a liposome is often referred to as “loading”. Exemplary methods are described in Lasic, et al., FEBS Lett., 312: 255-258, 1992, which is incorporated herein by reference. The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating an mRNA into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the mRNA contained therein and/or facilitate the delivery of mRNA to the target cell or tissue.

Suitable liposomes in accordance with the present invention may be made in various sizes. In some embodiments, provided liposomes may be made smaller than previously known mRNA encapsulating liposomes. In some embodiments, decreased size of liposomes is associated with more efficient delivery of mRNA. Selection of an appropriate liposome size may take into consideration the site of the target cell or tissue and to some extent the application for which the liposome is being made.

In some embodiments, an appropriate size of liposome is selected to facilitate systemic distribution of antibody encoded by the mRNA. In some embodiments, it may be desirable to limit transfection of the mRNA to certain cells or tissues. For example, to target hepatocytes a liposome may be sized such that its dimensions are smaller than the fenestrations of the endothelial layer lining hepatic sinusoids in the liver; in such cases the liposome could readily penetrate such endothelial fenestrations to reach the target hepatocytes.

Alternatively or additionally, a liposome may be sized such that the dimensions of the liposome are of a sufficient diameter to limit or expressly avoid distribution into certain cells or tissues.

A variety of alternative methods known in the art are available for sizing of a population of liposomes. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomes may be determined by quasi-electric light scattering (QELS) as described in Bloomfield, Ann. Rev. Biophys. Bioeng., 10:421-150 (1981), incorporated herein by reference. Average liposome diameter may be reduced by sonication of formed liposomes. Intermittent sonication cycles may be alternated with QELS assessment to guide efficient liposome synthesis.

### Therapeutic Use of Compositions

In one aspect, the present invention, among other things, provides a LNP formulations that encapsulate mRNA that is useful for therapeutic purposes. For example, in some embodiments, the LNP encapsulated mRNA encodes a protein that is deficient in a subject. For example, the mRNA may encode CFTR for treating cystitis fibrosis. Suitable mRNAs encoding CFTR are described, for example in WO 2020/106946 and PCT/US20/44158, each of which are incorporated herein by reference in their entirety. As another example, the mRNA may encode OTC for treating Ornithine Transcarbamylase Deficiency, described in, for example, WO 2017/218524 the contents of which are incorporated herein its entirety.

In some embodiments, the LNP encapsulated mRNA encodes a protein that encodes a vaccine antigen, such as a SARS-CoV-2 antigen. Such SARS-CoV-2 antigens are described in U.S. 63/021,319, the contents of which are incorporated herein by reference.

In some embodiments, the mRNA is codon optimized. Various codon-optimized methods are known in the art.

Gene Therapy

In some embodiments, the LNP formulation described herein are suitable for pharmaceutical composition comprising codon optimized nucleic acids encoding a protein that is used to treat subjects in need thereof. In some embodiments, a pharmaceutical composition comprising a rAAV vector described herein is used to treat subjects in need thereof. The pharmaceutical composition containing a rAAV vector or particle of the invention contains a pharmaceutically acceptable excipient, diluent or carrier. Examples of suitable pharmaceutical carriers are well known in the art and include phosphate buffered saline solutions, water, emulsions, such as oil/water emulsions, various types of wetting agents, sterile solutions and the like. The pharmaceutical composition can be in a lyophilized form. Such carriers can be formulated by conventional methods and are administered to the subject at a therapeutically effective amount.

The rAAV vector is administered to a subject in need thereof via a suitable route. In some embodiments, the rAAV vector is administered by intravenous, intraperitoneal, subcutaneous, or intradermal routes. In one embodiment, the rAAV vector is administered intravenously. In embodiments, the intradermal administration comprises administration by use of a “gene gun” or biolistic particle delivery system. In some embodiments, the rAAV vector is administered via a non-viral lipid nanoparticle. For example, a composition comprising the rAAV vector may comprise one or more diluents, buffers, liposomes, a lipid, a lipid complex. In some embodiments, the rAAV vector is comprised within a microsphere or a nanoparticle, such as a lipid nanoparticle or an inorganic nanoparticle.

In some embodiments, a rAAV is pseudotyped. A pseudotyped rAAV is an infectious virus comprising any combination of an AAV capsid protein and a rAAV genome. Pseudotyped rAAV are useful to alter the tissue or cell specificity of rAAV, and may be employed alone or in conjunction with non-pseudotyped rAAV to transfer one or more genes to a cell, e.g., a mammalian cell. For example, pseudotyped rAAV may be employed subsequent to administration with non-pseudotyped rAAV in a mammal which has developed an immune response to the non-pseudotyped rAAV. Capsid proteins from any AAV serotype may be employed with a rAAV genome which is derived or obtainable from a wild-type AAV genome of a different serotype or which is a chimeric genome, i.e., formed from AAV DNA from two or more different serotypes, e.g., a chimeric genome having 2 ITRs, each ITR from a different serotype or chimeric ITRs. The use of chimeric genomes such as those comprising ITRs from two AAV serotypes or chimeric ITRs can result in directional recombination which may further enhance the production of transcriptionally active intermolecular concatamers. Thus, the 5′ and 3′ ITRs within a rAAV vector of the invention may be homologous, i.e., from the same serotype, heterologous, i.e., from different serotypes, or chimeric, i.e., an ITR which has ITR sequences from more than one AAV serotype.

In some embodiments, the rAAV vector is an AAV1, AAV2, AAV3, AAV4, AAV5, AAV6, AAV7, AAV8, AAV9, AAV10, or AAV11 vector. In some embodiments, the rAAV vector is AAV1. In some embodiments, the rAAV vector is AAV2. In some embodiments, the rAAV vector is AAV3. In some embodiments, the rAAV vector is AAV4. In some embodiments, the rAAV vector is AAV5. In some embodiments, the rAAV vector is AAV6. In some embodiments, the rAAV vector is AAV7. In some embodiments, the rAAV vector is AAV8. In some embodiments, the rAAV vector is AAV9. In some embodiments, the rAAV vector is AAV10. In some embodiments, the rAAV vector is AAV11. In some embodiments, the rAAV vector is sequence optimized. In some embodiments, the rAAV capsid is modified. For example, in some embodiments, the rAAV8 capsid is modified.

### EXAMPLES

While certain compounds, compositions and methods of the present invention have been described with specificity in accordance with certain embodiments, the following examples serve only to illustrate the compounds of the invention and are not intended to limit the same.

### Example 1. Effect of Sugar, Buffer Ratio and pH on LNP Stability

Analyses were performed to assess the stability of LNPs in the presence of varying amounts of a sugar, here trehalose, varying buffer strengths, and/or varying pH levels. Collectively, the data from these studies show that at lower pH levels, higher minimum buffer strengths were required to maintain stability. Furthermore, the results also showed, that when the sugar, trehalose, is maintained at a constant percentage within the formulation, that as pH of the formulation goes up, the required minimum buffer strength to maintain LNP stability goes down.

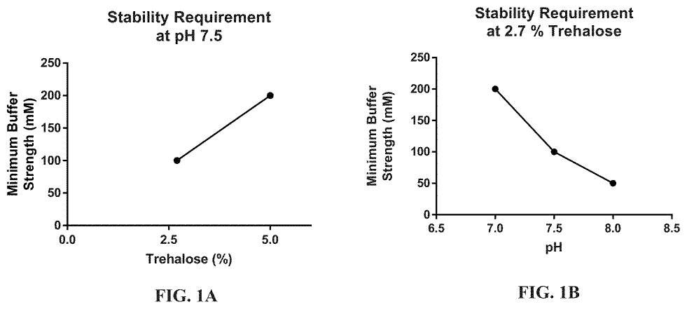

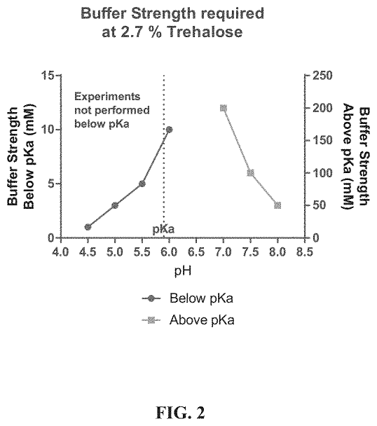

FIG. 1A is a graph that indicates that at pH 7.5, increasing the percentage of the sugar, trehalose in the LNP formulation, results in a concomitant increase in the minimum buffer strength required in the LNP formulation. FIG. 1B is a graph that shows when trehalose is maintained at a constant percentage (i.e., 2.7%), that as pH levels increase, the minimum buffer strength decreases.

What these data showed is that a lower sugar/buffer ratio is required at certain pHs. Furthermore, the data also show that the lower the pH in the LNP formulation, the higher the buffer strength is required to stabilize the LNP at certain sugar concentrations. For example, if the sugar concentration is maintained constant, the lower the pH level, the higher the buffer strength needed to maintain LNP stability.

### Example 2. Lowering Buffer Strength Results in Higher Stability Below pKa of the Lipid

Studies were performed to assess lipid pKa dependent behaviour. For these studies LNP formulations were analysed which were formulated to comprise 2.7% trehalose and pH 4.5 using citrate buffer. What these analyses showed, was that lowering the buffer strength resulted in higher stability of the LNP below the pKa of the lipid. Specifically, LNP stability was observed to decrease with increasing buffer strength tested, i.e., 1, 10, 20, 50, 75, to 100 mM. This is illustrated in graphical format in FIG. 2.

These data indicate that buffer strength is better at stabilizing LNP formulation following dilution of a sample. For example, stability was observed visually in the following scenarios: 1) 2.7% trehalose+100 mM Tris pH 7.5 (observation of solution—clear); 2) 2.7% trehalose+20 mM Tris pH 7.5+100 mM NaCl (observation of solution—crashed/cloudy); 3) 2.7% trehalose+16 mM Tris pH 7.5+220 mM NaCl (observation of solution—clear).

Collectively, from these data it was concluded that maintaining higher ionic strength was desirable to prevent LNP aggregation, and resultant mRNA stability. It was deduced that this could be achieved in various ways, for example by 1) having a high buffer strength (e.g., 100 mM or greater); 2) combining a low buffer strength (e.g., 15-20 mM) with a high salt concentration (e.g., 200 mM or greater); or combining a medium buffer strength (e.g., 40-50 mM) with a medium salt concentration (e.g., 50-100 mM).

### Example 3. Potency vs Stability

It has previously been observed that highly potent LNPs are associated with a higher amount of LNP aggregation and subsequent mRNA degradation. The LNP formulations described herein were investigated to determine whether these formulations had any impact on the ability to obtain LNPs encapsulated mRNA that are resistant to aggregation and to subsequent mRNA degradation.

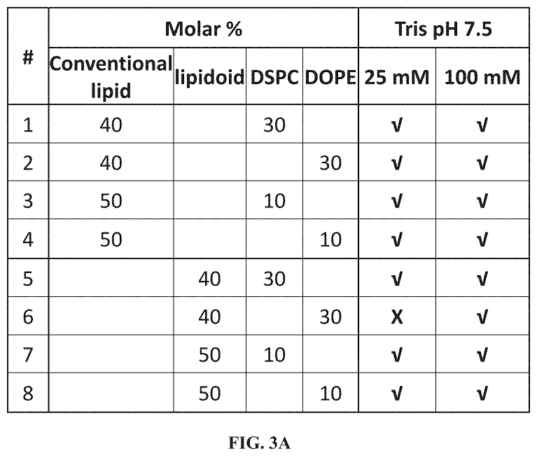

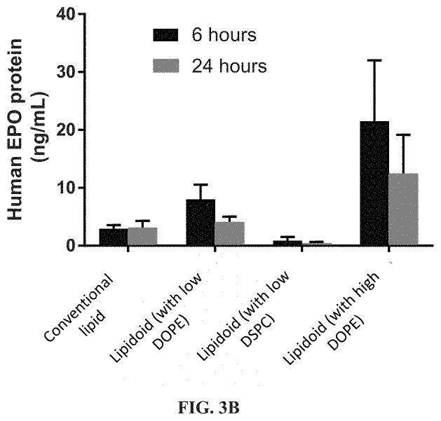

Various LNP formulations encapsulating human Erythropoietin (EPO) mRNA were tested for stability at 6 hours and 25 hours. The tested LNP formulations had previously been found to be prone to aggregation. As shown in FIGS. 3A and 3B, use of LNP formulations described herein allowed for the successful formulation of desirable, highly potent LNPs that were resistant to aggregation.

The different LNP formulations that were tested are depicted in FIG. 3A and in FIG. 3B. The data from FIG. 3B were from in vivo studies in which the described LNP formulations were analysed at either 6 hours or 24 hours after dosing in mice. The data show that expression of human EPO protein at both 6 hours and 24 hours when using highly potent lipids, including for example lipidoids with high concentration of DOPE.

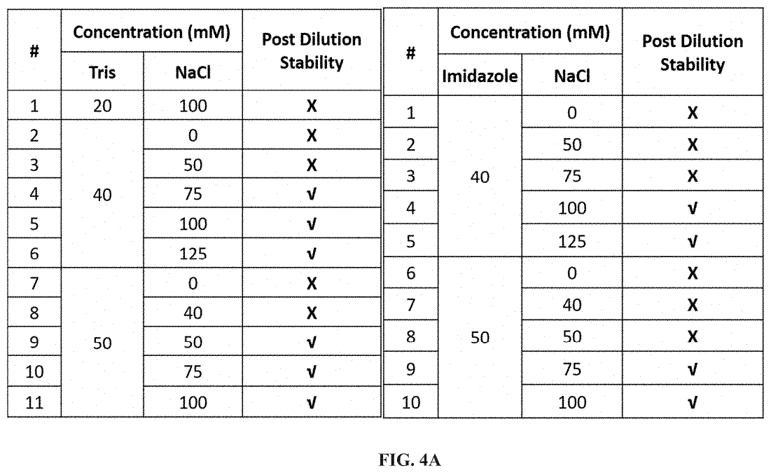

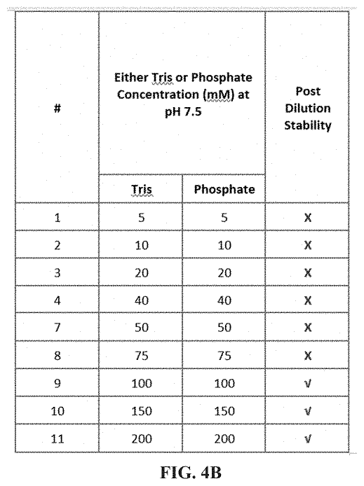

FIG. 4A shows various combinations of buffer and salt concentrations tested in the LNP formulations and resultant post-dilution stability associated with the various LNP formulations. The data are consistent with the results presented in Example 2, namely that higher ionic strength was desirable to prevent LNP aggregation, and resultant mRNA stability. In particular, these data confirmed that combining a medium buffer strength (e.g., 40-50 mM) with a medium salt concentration (e.g., 50-125 mM) resulted in a stable LNP formulation post dilution.

FIG. 4B shows a table that summarizes the stability of LNP formulations post dilution. For these assays, the LNPs varied only with respect to the Tris or Phosphate buffer concentrations. The LNPs in this study were all formulated in Tris or Phosphate buffer and 2.7% Trehalose. As the data show, formulation pH was reached at 20 mM buffer strength, however, these LNP formulations were not stable. The LNP formulations were stable when the buffer strength reached 100 mM or greater. The data are consistent with the results presented in Example 2, namely that higher ionic strength was desirable to prevent LNP aggregation, and resultant mRNA stability.

### Example 4. Effect of the Ratio of Sugar to Buffer on Encapsulation Efficiency and Size of Lipid Nanoparticles

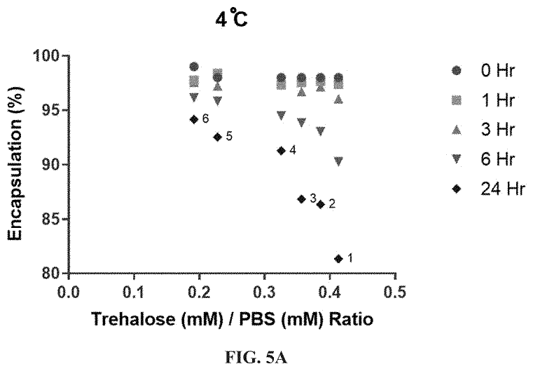

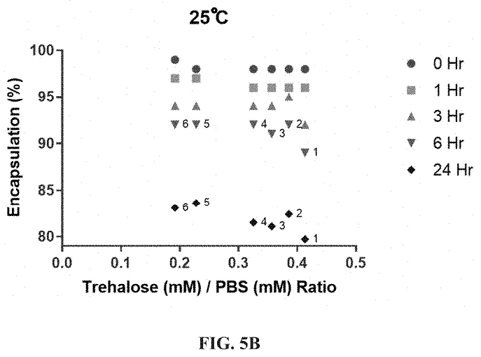

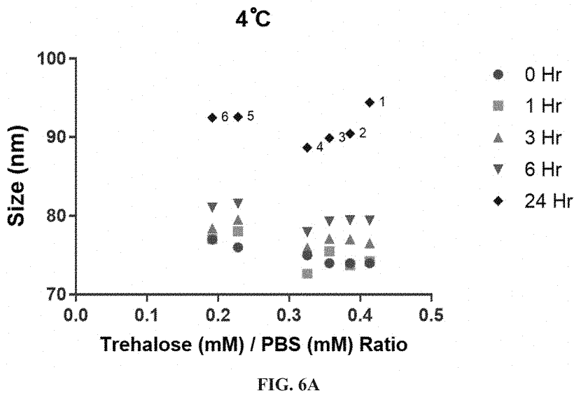

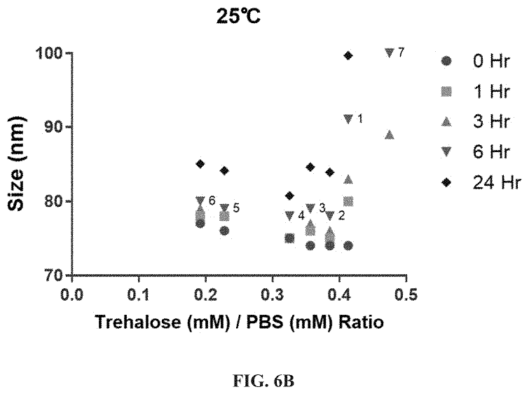

Studies were performed to assess the effect of the ratio of sugar to buffer on the stability of the formulation at −20° C. For these studies, LNP formulations were analysed which were formulated at a starting mRNA concentration of between 0.9 mg/ml to 1.6 mg/ml and comprising exemplary trehalose to PBS ratios of between 0.19 to 0.47 (Table 1). Encapsulation efficiencies (FIG. 5A and FIG. 5B) and sizes of the lipid nanoparticles (FIG. 6A and FIG. 6B) were evaluated at 4° C. and 25° C. at varying trehalose to PBS ratios of the LNP formulation. What these analyses showed, was that a lower trehalose to PBS ratio of the LNP formulation was beneficial in preventing a decrease in encapsulation and an increase in LNP size, thereby resulting in higher stability of the LNP formulation. Overall, LNP formulation stability was greater at low sugar to buffer ratio. This is illustrated in graphical format showing the effect of sugar to buffer ratio on encapsulation efficiencies (FIG. 5A and FIG. 5B) and LNP sizes (FIGS. 6A and 6B).

TABLE 1
LNP Formulations of varying trehalose to PBS ratios

| Exemplary | Starting | Starting | Starting | Final | Final | Final | Trehalose |
|---|---|---|---|---|---|---|---|
| Formulation | mRNA | trehalose | PBS | mRNA | trehalose | PBS | (mM)/PBS |
| No. | (mg/ml) | (%) | (X) | (mg/ml) | (%) | (X) | (mM) ratio |
| 1. | 1 | 10 | 2 | 0.3 | 3 | 1.4 | 0.413217 |
| 2. | 1 | 10 | 2 | 0.27 | 3 | 1.5 | 0.38567 |
| 3. | 1 | 10 | 2.2 | 0.3 | 2.7 | 1.46 | 0.356612 |
| 4. | 1 | 10 | 2.2 | 0.27 | 2.7 | 1.6 | 0.325409 |
| 5. | 1.6 | 10 | 2 | 0.3 | 1.89 | 1.6 | 0.227786 |
| 6. | 1.6 | 10 | 2 | 0.27 | 1.69 | 1.7 | 0.191701 |
| 7. | 0.9 | 10 | 2 | 0.3 | 3.3 | 1.34 | 0.474892 |

Encapsulation efficiencies were evaluated at various exemplary time points (0 hr, 1 hr, 3 hr, 6 hr and 24 hr) and the observed percent encapsulation efficiency is graphically depicted at 4° C. (FIG. 5A) and 25° C. (FIG. 5B). The results showed that in LNP formulations with increasing trehalose to PBS ratio, a decrease in encapsulation was observed, indicating decreased stability. The results were striking at 4° C. but a similar trend was observed at 25° C.

The LNP sizes were measured at various exemplary time points (0 hr, 1 hr, 3 hr, 6 hr and 24 hr) and the observed LNP size (in nanometers) is graphically depicted at 4° C. (FIG. 6A) and 25° C. (FIG. 6B). The results showed that in LNP formulations with increasing trehalose to PBS ratio, a decrease in encapsulation was observed, indicating decreased stability. The results were striking at 25° C. but a similar trend was observed at 4° C.

Overall, the results from these studies indicated that a low trehalose to PBS ratio favoured increased encapsulation and decreased LNP size, corresponding to higher stability of the LNPs.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A liquid lipid nanoparticle (LNP) formulation encapsulating mRNA encoding a peptide or polypeptide, that is resistant to aggregation and to mRNA degradation, the LNP formulation comprising:
a. one or more LNPs having a lipid component comprising or consisting of a cationic lipid, a non-cationic lipid, a PEG-modified lipid and optionally cholesterol;
b. mRNA encapsulated within the one or more lipid nanoparticles and encoding a peptide or polypeptide;
c. a sugar or a sugar alcohol;
d. an LNP formulation pH of from 6.0 to 8.0;
e. a pH buffer that at a minimum buffered ionic strength provides the LNP formulation pH;
f. optionally one or more additional agents that provide ionic strength to the LNP formulation;
wherein a total concentration of pH buffer from (e.), and optionally one or more additional agents from (f.), provide(s) an ionic strength of the LNP formulation that is at least two times greater than the minimum buffered ionic strength.

2. The LNP formulation of claim 1, wherein following at least three rounds of freezing at −20° C. and rethawing, the LNP formulation exhibits (i) less aggregation, (ii) less degradation of the encapsulated mRNA, or (iii) both (i) and (ii), as compared to an identical LNP formulation that has only the minimum buffered ionic strength in the LNP formulation instead of an ionic strength that is at least two times greater than the minimum buffered ionic strength.

3. The LNP formulation of claim 1, wherein the non-cationic lipid is selected from 1,2-Dierucoyl-sn-glycero-3-phosphoethanolamine (DEPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, or 1-stearoyl-2-oleoyl-phosphatidyethanolamine (SOPE).

4. The LNP formulation of claim 1, wherein the non-cationic lipid is dioleoylphosphatidylethanolamine (DOPE), and wherein the DOPE is at a lipid molar ratio of 10% or greater.

5. (canceled)

6. The LNP formulation of claim 1, wherein the cationic lipid is a lipidoid, wherein the lipidoid is a lipid molar ratio of 40%-60%.

7. (canceled)

8. The LNP formulation of any one of claims 1-4, wherein the mRNA encodes a vaccine antigen.

9. (canceled)

10. The LNP formulation of claim 1, wherein the sugar is a disaccharide, wherein the disaccharide is at a concentration of about 2.5-3.0% and wherein the disaccharide to buffer ratio is between 0.2-0.5.

11-14. (canceled)

15. The LNP formulation of claim 1, wherein the pH is between about 6.0 and about 8.0.

16-17. (canceled)

18. The LNP formulation of claim 1, wherein the buffer is selected from the group consisting of a phosphate buffer, a citrate buffer, an imidazole buffer, a histidine buffer, and a Good's buffer.

19-22. (canceled)

23. The LNP formulation of claim 1, wherein the minimum buffered ionic strength is between 100 mM-200 mM.

24. The LNP formulation of claim 1, wherein the one or more agents that provide ionic strength comprises a salt or a sugar, wherein the salt is selected from the group consisting of NaCl, KCl and CaCl₂, and wherein the sugar is trehalose.

25.-26. (canceled)

27. The LNP formulation of claim 1, wherein the total concentration of pH buffer is between about 15-250 mM.

28-30. (canceled)

31. The LNP formulation of claim 1, wherein the ionic strength of the LNP formulation is at least two times greater and less than 20 times greater than the minimum buffered ionic strength, and wherein the ionic strength of the LNP formulation is between about 150 mM -750 mM.

32-36. (canceled)

37. The LNP formulation of claim 1, wherein the LNPs have a diameter between about 70 nm-90 nm, and wherein the N/P ratio is between 3-5.

38. The LNP formulation of claim 1, wherein the lipid component comprises or consists of DMG-PEG-2000, cKK-E10, cholesterol, and DOPE.

39.-40. (canceled)

41. The LNP formulation of claim 1, wherein the mRNA is at a final concentration of between about 0.05 mg/mL and 1.0 mg/mL.

42. (canceled)

43. The LNP formulation of claim 1, wherein the LNPs are stable at −20° C. for at least 3 months, 6 months, 12 months, or more than 12 months.

44. The LNP formulation of claim 1, wherein the LNP formulation is stable following dilution.

45. The LNP formulation of claim 1, wherein subcutaneous or intramuscular delivery of the formulation is accompanied with reduced pain in comparison to a formulation that does not comprise a buffer having a concentration of or below 300 mM and a pH of between about 7.0 and 7.5.

46. (canceled)

47. A method of reducing LNP degradation and/or aggregation, the method comprising storing the LNP in a formulation comprising:
a. one or more LNPs having a lipid component comprising or consisting of a cationic lipid, a non-cationic lipid, a PEG-modified lipid and optionally cholesterol;
b. mRNA encapsulated within the one or more lipid nanoparticles and encoding a peptide or polypeptide;
c. a sugar or a sugar alcohol;
d. an LNP formulation pH of from 6.0 to 8.0;
e. a pH buffer that at a minimum buffered ionic strength provides the LNP formulation pH;
f. optionally one or more additional agents that provide ionic strength to the LNP formulation;
wherein a total concentration of pH buffer from (e.), and optionally one or more additional agents from (f.), provide(s) an ionic strength of the LNP formulation that is at least two times greater than the minimum buffered ionic strength.
